(19) United States
(12) Patent Application Publication
(10) Pub. No.: US 2025/0204847 A1
(43) Pub. Date: Jun. 26, 2025

(54) METHODS AND APPARATUS FOR MEASURING SKIN CHARACTERISTICS

(71) Applicant: Carruthers Instruments Inc., Vancouver (CA)

(72) Inventors: Jean CARRUTHERS, Vancouver (CA); Gyasi BOURNE, Vancouver (CA); Jeffrey DOYLE, Victoria (CA); Robert PRIOR, Sidney (CA); Zachary HARDAT, Victoria (CA); Matthew O'HAGAN, Victoria (CA); Cheyenne HEENAN, Sanich (CA); Simon PEARSON, Victoria (CA)

(21) Appl. No.: 19/079,351

(22) Filed: Mar. 13, 2025

Related U.S. Application Data
(63) Continuation of application No. PCT/CA2023/051225, filed on Sep. 14, 2023.
(60) Provisional application No. 63/407,159, filed on Sep. 16, 2022.

(51) Int. Cl. A61B 5/00

(52) U.S. Cl. CPC A61B 5/442; A61B 2562/0261

(57) ABSTRACT

A skin measuring apparatus measures skin characteristics. The apparatus comprises: a mover attached to a housing to allow the mover to move in a first direction and a second direction relative to the housing; a position sensor attached to the housing for measuring a position of the mover as the mover moves relative the housing between first and second positions; a force sensor for measuring a force applied to the mover; a latch fixed to the mover; a releasable catch attached to the housing, the releasable catch engageable with the latch to prevent movement of the mover in the second direction from the second position until the releasable catch is released. The first direction is opposite the second direction; a first end of the mover is releasably attachable to a skin sample.

### REFERENCE TO RELATED APPLICATIONS

This application is a continuation of Patent Cooperation Treaty (PCT) application No. PCT/CA2023/051225 having an international filing date of 14 Sep. 2023 which in turn claims priority from, and for the purposes of the United States the benefit under 35 USC 119 in relation to, U.S. patent application No. 63/407,159 filed 16 Sep. 2022. All of the applications referred to in this paragraph are hereby incorporated herein by reference.

### TECHNICAL FIELD

This invention relates to the field of skin care, and in particular to methods and apparatus for measuring and/or determining one or more skin characteristics.

### BACKGROUND

Human beings have adopted a range of practices to improve skin health, skin appearance and relieve various skin conditions. These practices include dieting, avoiding sun exposure and the use of various products and services, such as, cosmetics, botulinum, exfoliation, fillers, laser resurfacing, microdermabrasion, peels, retinol therapy, ultrasonic skin treatment, etc. However, there remains a need to quantitatively measure skin characteristics to, for example, facilitate determining which practices should be used and/or which practices are effective.

The foregoing examples of the related art and limitations related thereto are intended to be illustrative and not exclusive. Other limitations of the related art will become apparent to those of skill in the art upon a reading of the specification and a study of the drawings.

### SUMMARY

The following embodiments and aspects thereof are described and illustrated in conjunction with systems, tools and methods which are meant to be exemplary and illustrative, not limiting in scope. In various embodiments, one or more of the above-described problems have been reduced or eliminated, while other embodiments are directed to other improvements.

Non-limiting aspects of the invention include the following.

One aspect of the invention provides a skin measuring apparatus for measuring skin characteristics, the skin measuring apparatus comprising: a mover attached to a housing to allow the mover to move in a first direction and a second direction relative to the housing; a position sensor attached to the housing for measuring a position of the mover as the mover moves relative the housing between first and second positions; a force sensor for measuring a force applied to the mover; a latch fixed to the mover; a releasable catch attached to the housing, the releasable catch engageable with the latch to prevent movement of the mover in the second direction from the second position until the releasable catch is released. The first direction is opposite the second direction. A first end of the mover is releasably attachable to a skin sample.

A mover attached to a housing may allow the mover to rotate about a first axis in the first direction and the second direction relative to the housing.

The mover may be rotatable in the first direction and/or the second direction by actuation of a rotary actuator or motor attached to the mover.

The releasable catch may be attached to a catch body, the catch body rotatable about the first axis.

The force sensor may be fixed relative to the housing. A sensing portion of the force sensor may be attached to the catch body to measure a torque about the first axis, the torque applied to the catch body by the latch.

An angular difference between the first position and the second position may be between approximately 10° and 180°.

An angular difference between the first position and the second position may be between approximately 5° and 70°.

An angular difference between the first position and the second position may be between approximately 10° and 25°.

The mover may be rotatably attached to the housing by one or more bearings.

The mover may be rotatably attached to the housing by one or more bushings.

The mover may be attached to a housing to allow the mover to translate along a first axis in the first direction and the second direction relative to the housing.

The mover may be translatable in the first direction and/or the second direction by actuation of an actuator or motor attached to the mover.

A distance between the first position and the second position may be between approximately 5 mm and 50 mm.

A distance between the first position and the second position may be between approximately 10 mm and 30 mm.

The mover may be translatably attached to the housing by one or more rails.

The mover may be translatably attached to the housing by one or more tracks.

A sensing portion of the force sensor may be fixed to the mover.

The latch may engage the releasable catch by resilient deformation of one or more of the latch and the catch.

The latch may engage the releasable catch by restorative deformation of one or more of the latch and the catch.

The catch may be resiliently deformable to thereby release the latch by application of an external force to a trigger.

The trigger may be accessible through an aperture defined by the housing.

The apparatus may comprise a block releasably engageable with the latch to hold the mover at the first position by abutment of a first portion of the block with the housing and abutment of a second portion of the block with the latch.

Abutment of the second portion of the block with the latch may comprise receiving the latch within a slot defined by the block.

Abutment of the first portion of the block with the housing may comprise inserting at least a portion of the block into an aperture defined by the housing.

The apparatus may comprise a second releasable catch attached to the housing. The second releasable catch may be engageable with the latch to prevent movement of the mover from the first position until the second releasable catch is released.

The apparatus may comprise an adhesive for attaching the first end of the mover to the skin sample.

The position sensor may comprise a Hall effect sensor fixed relative to the housing and a magnet fixed relative to the mover.

The releasable catch may be spring-loaded.

The housing may surround at least a portion of the mover.

The force sensor may comprise a torque sensor.

The force sensor may comprise a strain gauge.

The apparatus may comprise a stand. The stand may comprise: a base; an arm extending in a z-direction from the base, wherein the housing is attached to the arm to support the housing above the base; and one or more holders supported by the base, the one or more holders defining a cradle to receive a body part of the skin sample.

The apparatus may comprise a platform adjustably attached to the base, wherein the one or more holders are attached to the platform.

A position and/or orientation of the platform relative to the base may be adjustable to accommodate a shape of the body part.

A y-direction position and/or an orientation about an x-direction axis of the platform relative to the base may be adjustable to accommodate a shape of the body part, wherein the x, y and z-directions are mutually orthogonal.

The platform may be fixed to a slider attached a rail attached to the base to allow the platform to slide in the y-direction on the rail relative to the base.

A rail clamp may be selectively engageable to prevent relative movement between the platform and the rail.

The platform may be fixed to the slider by a hinge to allow rotation of the platform about an x-direction hinge axis.

A z-direction distance between the housing and the base may be adjustable by selectively releasing and locking a height clamp.

Positions and/or orientations of the one or more holders may be adjustable to vary the size of the cradle.

Another aspect of the invention provides a method for measuring skin characteristics. The method comprises: providing a mover attached to a housing to allow the mover to move in a first direction and a second direction relative to the housing; setting a first position of the mover; adhering a first end of the mover to a skin sample; moving the mover in the first direction to a second position; releasing the mover to allow the mover to move in the second direction; measuring a force applied to the mover by the skin sample; measuring the position of the mover while it moves in the first and second directions; determining one or more skin characteristics based at least in part on the measured force and the measured angular position.

Moving the mover in the first direction to the second position may comprise rotating the mover about a first axis of the mover in the first direction to the second position.

An angular difference between the first position and the second position may be between approximately 5° and 70°.

An angular difference between the first position and the second position may be between approximately 10° and 25°.

Moving the mover in the first direction to the second position may comprise actuating a rotary actuator connected to the mover.

Rotating the mover in the first direction to achieve the second position of the mover may comprise rotating the mover in the first direction until a latching mechanism is engaged to prevent rotation of the mover in the second direction.

The releasable catch may be attached to a catch body. The catch body may be rotatable about the longitudinal axis of the mover. Measuring the force applied to the mover by the skin sample may comprise measuring a force about the longitudinal axis of the mover applied to the catch body by the latch.

Moving the mover in the first direction to the second position may comprise translating the mover in the first direction to the second position.

Moving the mover in the first direction to the second position may comprise translating the mover in the first direction by actuation of an actuator or motor attached to the mover.

A distance between the first position and the second position may be between approximately 5 mm and 50 mm.

A distance between the first position and the second position may be between approximately 10 mm and 30 mm.

The mover may be translatably attached to the housing by one or more rails.

The mover may be translatably attached to the housing by one or more tracks.

Setting the first position of the mover may comprise releasably engaging a block with a latch fixed to the mover to hold the mover at the first position by abutting a first portion of the block with the housing and abutting of a second portion of the block with the latch.

Abutting the second portion of the block with the latch may comprise receiving the latch within a slot defined by the block.

Abutting the first portion of the block with the housing may comprise inserting at least a portion of the block into an aperture defined by the housing.

Adhering a first end of the mover to a skin sample may comprise employing an adhesive to adhere the first end of the mover to the skin sample.

Moving the mover in the first direction to the second position may comprise engaging a latching mechanism by engaging a latch fixed to the mover with a catch fixed to the housing.

Engaging the latching mechanism may comprise engaging a latch fixed to the mover with a catch fixed to the housing by resiliently deforming one or more of the latch and the catch.

Engaging the latching mechanism may comprise engaging a latch fixed to the mover with a catch fixed to the housing by restoratively deforming one or more of the latch and the catch.

Releasing the mover to allow the mover to move in the second direction may comprise resiliently deforming the catch to thereby release the latch by applying an external force to a trigger attached to the catch.

The trigger may be accessible through an aperture defined by the housing.

Aspects of the invention provide methods comprising any features, combinations of features and/or sub-combinations of features described herein or inferable therefrom.

Aspects of the invention provide apparatus comprising any features, combinations of features and/or sub-combinations of features described herein or inferable therefrom.

It is emphasized that the invention relates to all combinations of the above features, combinations of the above features and/or sub-combinations of the above features even if these are recited in different claims or aspects.

In addition to the exemplary aspects and embodiments described above, further aspects and embodiments will become apparent by reference to the drawings and by study of the following detailed descriptions.

### BRIEF DESCRIPTION OF THE DRAWINGS

Exemplary embodiments are illustrated in referenced figures of the drawings. It is intended that the embodiments and figures disclosed herein are to be considered illustrative rather than restrictive.

FIG. 1 is an isometric view of a skin measuring apparatus for measuring one or more characteristics of skin according to an exemplary embodiment of the invention.

FIG. 2 is a schematic diagram of the skin measuring apparatus of FIG. 1.

FIG. 3 is an isometric view of a portion of the skin measuring apparatus of FIG. 1.

FIG. 4 is an isometric view of another portion of the skin measuring apparatus of FIG. 1.

FIG. 5A is a schematic diagram of a latching mechanism of a skin measuring apparatus according to an exemplary embodiment of the invention. FIG. 5B is another schematic diagram of the latching mechanism of FIG. 5A. FIG. 5C is another schematic diagram of the latching mechanism of FIG. 5A.

FIG. 6A is a schematic view of a latching mechanism of a skin measuring apparatus according to an exemplary embodiment of the invention. FIG. 6B is a top view of a portion of the latching mechanism of FIG. 6A.

FIG. 7A is a view of another latching mechanism of a skin measuring apparatus according to an exemplary embodiment of the invention. FIG. 7B is a bottom view of a portion of the latching mechanism of FIG. 7A.

FIG. 8A is a schematic diagram of a skin measuring apparatus according to another exemplary embodiment of the invention. FIG. 8B is a schematic diagram of the mover of the skin measuring apparatus of FIG. 8A in a first position. FIG. 8C is a schematic diagram of the mover of the skin measuring apparatus of FIG. 8A in a second position.

FIG. 9A is an isometric view of a stand for supporting a skin measuring apparatus for measuring one or more characteristics of skin according to an exemplary embodiment of the invention. FIG. 9B is a front view of the stand of FIG. 9A.

FIG. 10A is a schematic depiction of the stand of FIG. 9A in a first configuration. FIG. 10B is a schematic depiction of the stand of FIG. 9B in a second configuration.

FIG. 11 depicts a method for determining one or more skin characteristics according to an exemplary embodiment of the invention.

FIG. 12A is a schematic diagram of a first position of a shaft employed in a method for determining one or more skin characteristics according to an exemplary embodiment of the invention. FIG. 12B is a schematic diagram of a second position of the shaft of FIG. 12A. FIG. 12C is a schematic diagram of a third position of the shaft of FIG. 12A.

FIG. 13A is a schematic diagram of a first position of a body employed in a method for determining one or more skin characteristics according to an exemplary embodiment of the invention. FIG. 13B is a schematic diagram of a second position of the body of FIG. 12A.

FIG. 14 is a plot of angular position of a shaft employed in a method for determining one or more skin characteristics according to an exemplary embodiment of the invention as a function of time.

FIG. 15 is a schematic diagram of a portion of a skin measuring apparatus according to an exemplary embodiment of the invention.

### DESCRIPTION

Throughout the following description specific details are set forth in order to provide a more thorough understanding to persons skilled in the art. However, well known elements may not have been shown or described in detail to avoid unnecessarily obscuring the disclosure. Accordingly, the description and drawings are to be regarded in an illustrative, rather than a restrictive, sense.

One aspect of the invention provides a skin measuring apparatus for measuring one or more characteristics of a skin sample. The skin sample may comprise any suitable patch of skin on a body (e.g. a human body). The measurement of the one or more characteristics may occur in situ, such that the skin sample does not need to be removed from the body. The skin measuring apparatus may comprise a mover having a first end releasably attachable to the skin sample. Due to the attachment between the mover and the skin sample, the skin sample may be deformed by controllably moving (e.g. rotating or translating) the mover. The mover may be a shaft. The shaft may be rotatable about its longitudinal axis. As the shaft rotates about its longitudinal axis, the skin sample attached to the first end of the shaft may be deformed (e.g. twisted). Alternatively, the mover may be a body translatable to deform the skin in a linear manner. The skin measuring apparatus may comprise one or more sensors to directly or indirectly measure one or more characteristics of the skin as the mover is moved (e.g. as the shaft is rotated or the body translated). For example, the skin measuring apparatus may comprise a position sensor that measures the position of the mover (e.g. angular position of the shaft as it rotates about its longitudinal axis or linear position of the body as it translates). As another example, the skin measuring apparatus may comprise a force (or torque) sensor which measures a force (or torque) applied to the mover by the skin. In some embodiments, the mover is moved by a set amount (e.g. between a first position and a second position) when the measuring apparatus is employed (e.g. to produce consistent, repeatable and comparable results). For example, where the mover comprises a rotatable shaft, the shaft may be rotated by a set amount (e.g. between a first position and a second position). To facilitate achieving the desired amount of movement (e.g. rotation or translation), one or more mechanisms may be provided to facilitate repeatedly setting the mover to the first position. The skin measuring apparatus may also comprise one or more mechanisms to releasably hold the mover at the second position when the mover is moved (e.g. rotated or translated) to the second position (or just past the second position). The skin measuring apparatus may comprise one or more release mechanisms to allow the mover to freely move from the second position (e.g. due to restorative deformation forces of the skin sample).

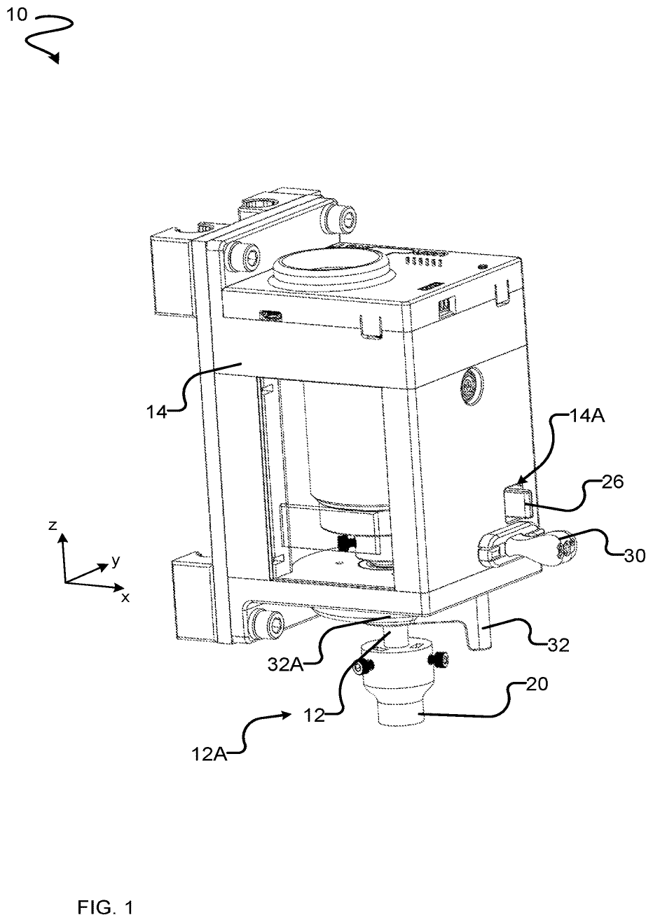

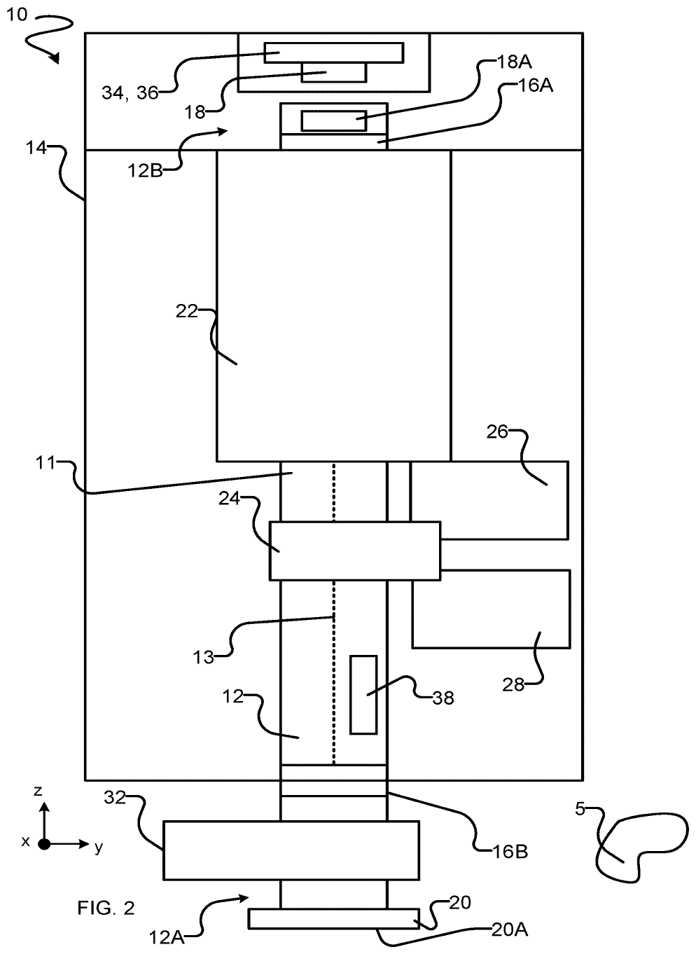

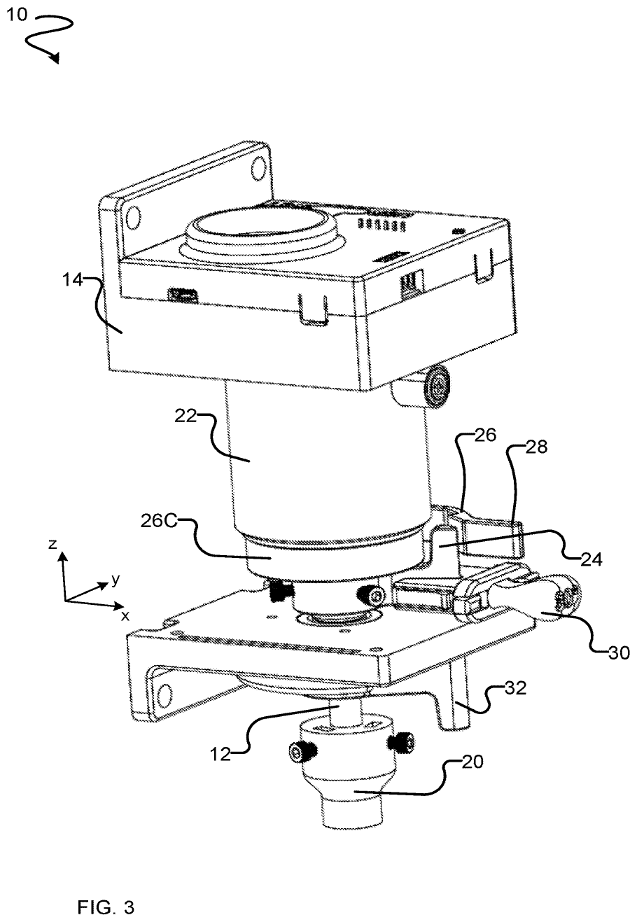

FIG. 1 shows an exemplary skin measuring apparatus 10 for measuring one or more skin characteristics according to an example embodiment of the invention. FIG. 2 shows a schematic representation of the FIG. 1 skin measuring apparatus 10 to better illustrate some of the features of skin measuring apparatus 10. FIG. 3 shows skin measuring apparatus 10 with a portion of the housing 14 (discussed further below) removed to better illustrate elements of skin measuring apparatus 10 that would otherwise by obscured by housing 14.

Skin measuring apparatus 10 comprises a mover 11 releasably attachable to a skin sample 5. Mover 11 comprises a longitudinally extending shaft 12 rotatably attached to a housing 14 such that shaft 12 remains free to rotate about its longitudinal axis 13 relative to housing 14 (e.g. housing 14 may remain stationary while shaft 12 rotates about its longitudinal axis 13) in a first direction 2 and a second direction 4 (opposite first direction 2). Shaft 12 may be attached to housing 14 by one or more bearings, bushings or the like to allow shaft 12 to rotate about its longitudinal axis 13 relative to housing 14. For example, in the illustrated embodiment, first and second bearings 16A, 16B are provided to allow shaft 12 to rotate relative to housing 14. Shaft 12 may otherwise be fixed relative to housing (e.g. in the x, y, and z directions and/or about the y and z-direction axes of shaft 12).

Housing 14 may surround at least a portion of shaft 12, although this is not mandatory. In some embodiments, housing 14 comprises a support for other elements of skin measuring apparatus 10 (as discussed further herein) without surrounding shaft 12 or providing any enclosure around shaft 12 (or any portion thereof).

A first end 12A of shaft 12 may be releasably attachable to skin sample 5. Skin sample 5 may comprise any suitable patch of skin on a body (e.g. a human body). Skin measuring apparatus 10 may be used in situ, such that skin sample 5 does not need to be removed from the body. First end 12A may be attachable to skin sample 5 directly or indirectly by any suitable fastening means such as, for example, a suitable adhesive (e.g. doubled sided tape, cyanoacrylate adhesive, silicone adhesive, rosin adhesive, etc.). In this way, shaft 12 may be removed from skin sample as desired by releasing the adhesive (e.g. with suitable force, solvents, etc.).

In some embodiments, a probe 20 is provided at first end 12A of shaft 12. Probe 20 may have a probe surface 20A with a larger surface area for contact with skin sample 5 to increase adhesion between skin measuring apparatus 10 and skin sample 5. In some embodiments, probe surface 20A is flat, although this is not mandatory. Probe surface 20A may be rounded or curved. Probe surface 20A may have, for example, a surface area of between approximately 50 mm² and 200 mm².

Probe 20 may be releasably attached to first end 12A of shaft 12 such that probe 20 may be removed after use (e.g. for sanitary purposes, sterilization purposes, etc.) or interchanged with different probes 20 (e.g. of different sizes, shapes, textures, and/or the like) as desired. Probe 20 may be attached to first end 12A of shaft 12 using any suitable fastening means. For example, probe 20 may be threaded onto first end 12A, attached using one or more set screws, by adhesive, by a cotter pin, by friction fit, by magnetic attraction, etc.

Skin measuring apparatus 10 may comprise a position sensor 18 for determining an angular position of shaft 12 about its longitudinal axis 13. Position sensor 18 may comprise any suitable sensor for determining an angular position of shaft 12 about its longitudinal axis 13. Position sensor 18 may comprise a rotary encoder such as, not limited to, a mechanical absolute encoder, an optical absolute encoder, a magnetic absolute encoder, a capacitive absolute encoder, a geared multi-turn encoder, an incremental encoder, etc. In some embodiments, position sensor 18 is fixed to housing 14 and position sensor 18 tracks a target 18A attached to shaft 12 (e.g. at a second end 12B of shaft 12). For example, in some embodiments, position sensor 18 comprises a magneto-resistive sensor or Hall Effect sensor fixed to housing 14 that tracks the position of one or more magnetic targets 18A attached to the second end 12B of shaft 12. Position sensor 18 may comprise a camera (e.g. a video camera) for optical analysis of the angular position of shaft 12 or a target or marking on shaft 12.

Skin measuring apparatus 10 may comprise a force sensor 22 for measuring a force applied to shaft 12 (e.g. in the x or y-direction or about longitudinal axis 313) and/or for measuring/determining a resultant torque applied to shaft 12. Force sensor 22 may comprise any suitable sensor for measuring a force applied to shaft 12 (e.g. by skin sample 5). Force sensor 22 may measure the force applied to shaft 12 (e.g. by skin sample 5) directly or indirectly. Force sensor 22 may comprise a torque sensor. Force sensor may comprise a torque sensor (e.g. a rotary torque transducer) fixed to shaft 12 such that force sensor 22 rotates with shaft 12. Alternatively, force sensor 22 may comprise a torque sensor (e.g. a reaction torque sensor) fixed to housing 14 and in contact (directly or indirectly) with shaft 12, as discussed further herein. Force sensor 22 may comprise a strain gauge wherein a force applied to shaft 12 is then determined based at least in part on the output of the strain gauge. The output of force sensor 22 may be employed to determine a torque applied to shaft 12 (e.g. by skin sample 5).

In practice, it may be desirable to control the amount of rotation of shaft 12 when skin measuring apparatus 10 is employed to measure one or more characteristics of skin sample 5. Controlling the amount of rotation of shaft 12 may facilitate achieving consistent, reliable and/or comparable results from skin measuring apparatus 10. Skin measuring apparatus 10 may comprise one or more features to facilitate achieving a desired amount of rotation of shaft 12. In some embodiments, skin measuring apparatus 10 comprises a mechanism to control the amount of rotation of shaft 12 directly while allowing the rotation to start from any desirable angular position of shaft 12. In some embodiments, the amount of rotation of shaft 12 is controlled by limiting the rotation to rotation between set first and second positions 12-1, 12-2 of shaft 12.

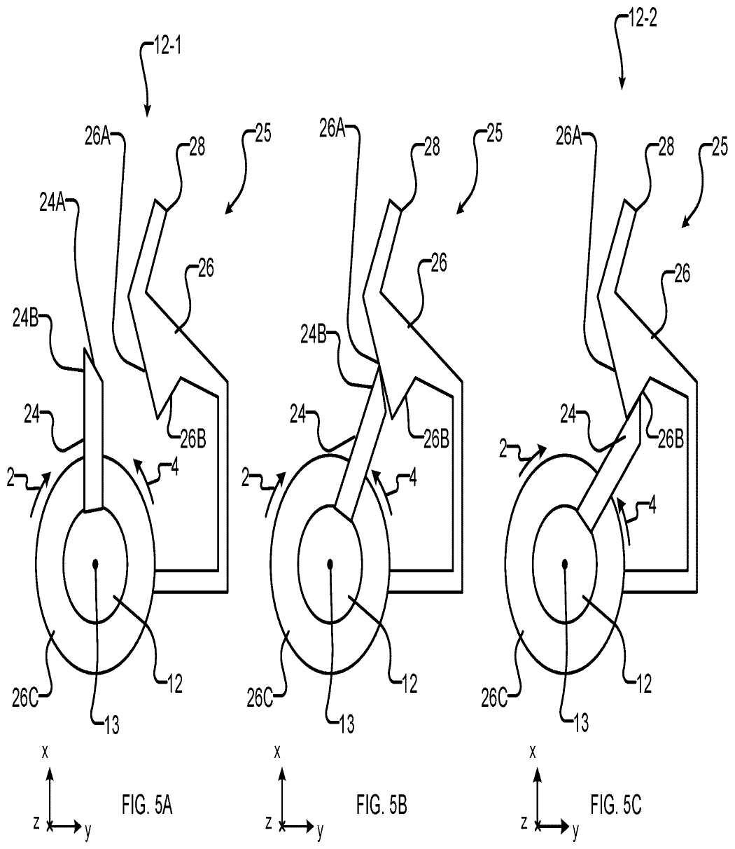

In some embodiments, skin measuring apparatus 10 comprises one or more features to facilitate setting skin measuring apparatus at a first position 12-1 (e.g. a starting position as shown, for example, in FIG. 5A) and/or one or more features to facilitate setting skin measuring apparatus at a second position 12-2 (e.g. a taut position as shown, for example, in FIG. 5C). Such features may comprise, for example, one or more of visual indicators, a detent mechanism, a mechanical latching mechanism, an electro-mechanical mechanism, a magnetic mechanism, an electro-magnetic mechanism, etc.

In some embodiments, skin measuring apparatus 10 comprises a selectively releasable latching mechanism 25. Latching mechanism 25 may comprise any suitable latching mechanism to facilitate holding shaft 12 at second position 12-2. Latching mechanism 25 may comprise a spring-loaded catch, a manual catch or latch, a ratcheting mechanism, a magnetic mechanism, an electromagnetic mechanism, an electromechanical mechanism, etc. Latching mechanism 25 may facilitate achieving a desired amount of rotation of shaft 12. For example, latching mechanism 25 may facilitate achieving second position 12-2 when rotating shaft 12 in first direction 2. By engaging latching mechanism 25, latching mechanism 25 may prevent undesired rotation of shaft 12 in second direction 4 from second position 12-2 until latching mechanism is disengaged. While first direction 2 is depicted as being clockwise and second direction 4 is depicted as being counter-clockwise, this is not mandatory. Instead, first direction 2 could be counter-clockwise while second direction 4 would be clockwise.

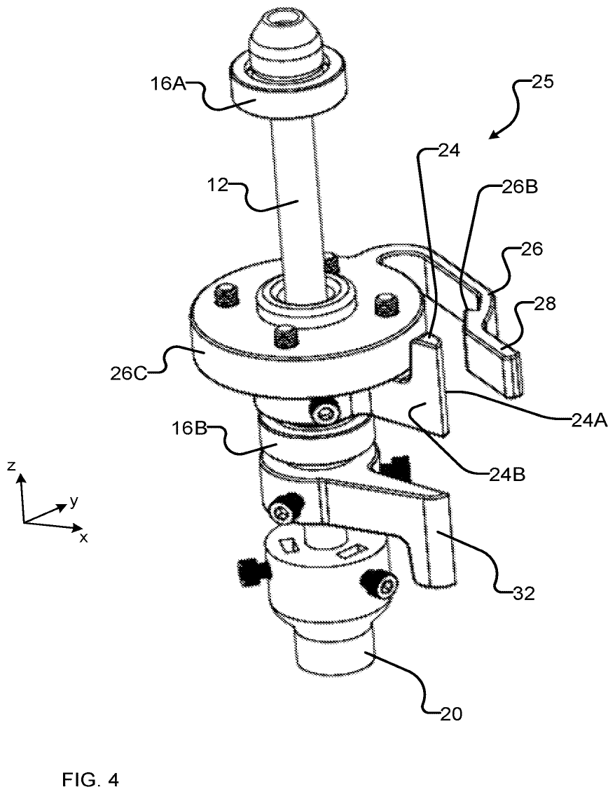

FIG. 4 depicts a portion of skin measuring apparatus 10 including shaft 12 and latching mechanism 25. It should be understood that other elements of skin measuring apparatus 10 are omitted from FIG. 4 to better illustrate latching mechanism 25.

Latching mechanism 25 comprises a latch 24 fixed to (or fixable to) shaft 12 such that latch 24 moves (e.g. rotates) with shaft 12 when shaft 12 is rotated about longitudinal axis 13. In some embodiments, at least a portion of latch 24 extends away from shaft 12 such that latch 24 protrudes from the surface of shaft 12 in the x and/or y-directions. In some embodiments, latch 24 comprises an indent or recess in shaft 12.

Latching mechanism 25 comprises a catch 26 to releasably engage latch 24 when latch 24 is rotated in first direction 2 to second position 12-1 (or just past second position 12-1). Catch 26 may be directly or indirectly fixed to housing 14. When catch 26 engages latch 24, catch 26 prevents rotation of latch 24 in second direction 4 from second position 12-2. In some embodiments, catch 26 automatically engages latch 24 when latch 24 is rotated past second position 12-2 in first direction 2. In some embodiments, catch 26 is manually engaged with latch 24 when latch 24 is rotated past second position 12-2 in first direction 2 (e.g. a user may manually move catch 26 into engagement with latch 24). Latch 24 and catch 26 may comprise any suitable latches and catches.

FIGS. 5A to 5C are schematic depictions of an exemplary latching mechanism 25 as latch 24 rotates from an unlatched configuration (FIG. 5A) to a latched configuration (FIG. 5C) according to an example embodiment of the invention. For simplicity, other elements of skin measuring apparatus 10 have been omitted from FIGS. 5A to 5C to better illustrate the function of latching mechanism 25.

As shaft 12 rotates in first direction 2, latch 24 also rotates until a leading edge 24A of latch 24 contacts a beveled edge 26A of catch 26, as shown in FIG. 5B. If shaft 12 continues to rotate (e.g. by application of an external force to shaft 12), contact of leading edge 24A and beveled edge 26A causes resilient deformation of catch 26 (and/or latch 24) to allow latch 24 to rotate past a hook 26B of catch 26. After latch 24 has rotated past hook 26B, catch 26 (and/or latch 24) begins to restoratively deform or snap back, at least partially, toward its non-deformed shape (e.g. due to the restorative deformation forces associated with the resilient deformation of catch 26). When catch 26 has restoratively deformed or snapped back (at least partially) toward its non-deformed shape, abutment of a trailing edge 24B of latch 24 and hook 26B of catch 26 holds shaft 12 in second position 12-2 and prevents rotation of latch 24 (and shaft 12) in second direction 4, as shown in FIG. 5C.

In some embodiments, engagement of latch 24 and catch 26 is releasable by applying external force (e.g. with a finger, an actuator, etc.) to a trigger 28. Trigger 28 may be attached to catch 26 such that by applying external force to trigger 28, catch 26 is resiliently deformed to thereby allow latch 24 to rotate past hook 26B in the second direction 4 thereby disengaging latching mechanism 25 and allowing shaft 12 to rotate in second direction 2.

In some embodiments, a force or torque applied to shaft 12 by skin sample 5 is determined indirectly by measuring a force applied by latch 24 on catch 26. For example, in some embodiments, force sensor 22 is connected to catch 26 to thereby measure the force applied by latch 24 on catch 26.

In some embodiments, catch 26 extends from a catch body 26C which is rotatably attached to shaft 12 such that force or torque applied to catch 26 (e.g. by latch 24) causes a torque about longitudinal axis 13. By attaching catch body 26C to a sensing part of force sensor 22 and by fixing force sensor 22 relative to housing 14 (e.g. by fixing force sensor to housing 14 directly or indirectly), force sensor 22 may measure the force (e.g. torque) applied to catch body 26C about longitudinal axis 13. Since the force applied to catch body 26C is indirectly caused by the force applied to shaft 12 by skin sample 5, force sensor 22 may, in this way, indirectly measure the force applied to shaft 12 by skin sample 5.

By measuring the force applied to shaft 12 by skin sample 5 indirectly rather than positioning a torque sensor between skin sample 5 and shaft 12, it may be possible to reduce interference with measurements taken by position sensor 18 that could otherwise be caused by positioning a force sensor between skin sample 5 and shaft 12.

In some embodiments, to set shaft 12 at first position 12-1, a catch 30 is provided to engage latch 24. In some embodiments, catch 30 is manually engaged to set shaft 12 to first position 12-1. In some embodiments, catch 30 is automatically engaged to set shaft 12 to first position 12-1. In some embodiments, catch 30 is similar to catch 26 except in that it holds latch 24 at first position 12-1 rather than at second position 12-2. In some embodiments, catch 30 engages latch 24 to prevent first direction 2 rotation of shaft 12. In some embodiments, catch 30 engages latch 24 to prevent first direction 2 rotation and/or second direction 4 rotation of shaft 12.

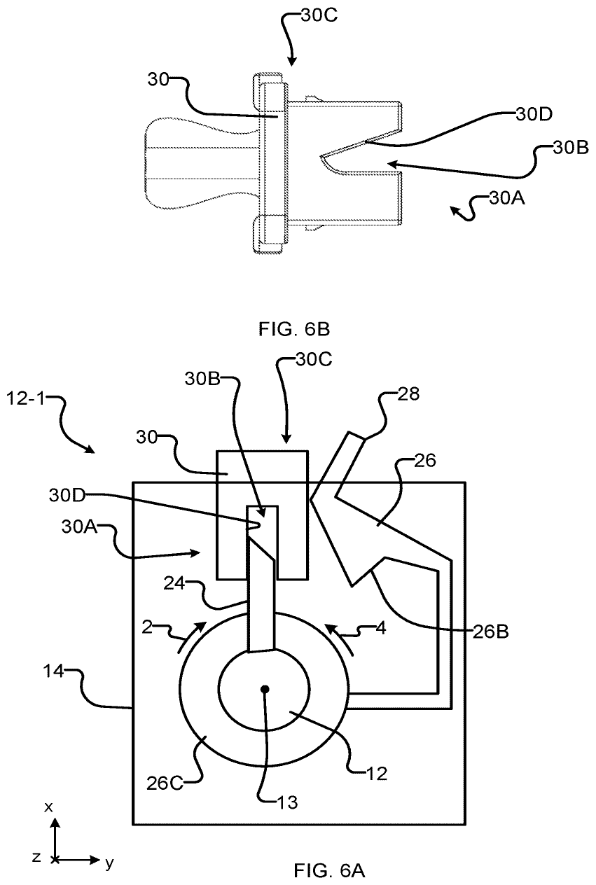

FIG. 6A shows a catch 30 engaging a latch 24 according to an example embodiment of the invention. FIG. 6B is a schematic depiction of a catch 30 according to an exemplary embodiment of the invention. A first end 30A of catch 30 defines a slot 30B that is at least partially complementary in shape to latch 24 such that at least a portion of latch 24 may be received within slot 30B. A second portion 30C of catch 30 may be shaped to engage housing 14 to prevent relative y and z-direction movement between catch 30 and housing 14. For example, a cross-section of second portion 30C of catch 30 may be complementary in shape to an aperture 14A (see FIG. 1) defined by housing 14 such that first end 30A of catch 30 can be pushed through aperture 14A to: (1) engage slot 30B with latch 24; and (2) engage second portion 30C of catch 30 with aperture 14A in housing 14 to thereby fix catch 30 relative to housing 14 (e.g. in the y and z-directions). When catch 30 is in such a position, abutment of latch 24 with an inner surface 30D of slot 30B prevents first direction 2 and second direction 4 rotation of shaft 12 thereby fixing shaft 12 in first position 12-1. Catch 26 can be disengaged from latch 24 by manually removing catch 30 from aperture 14A.

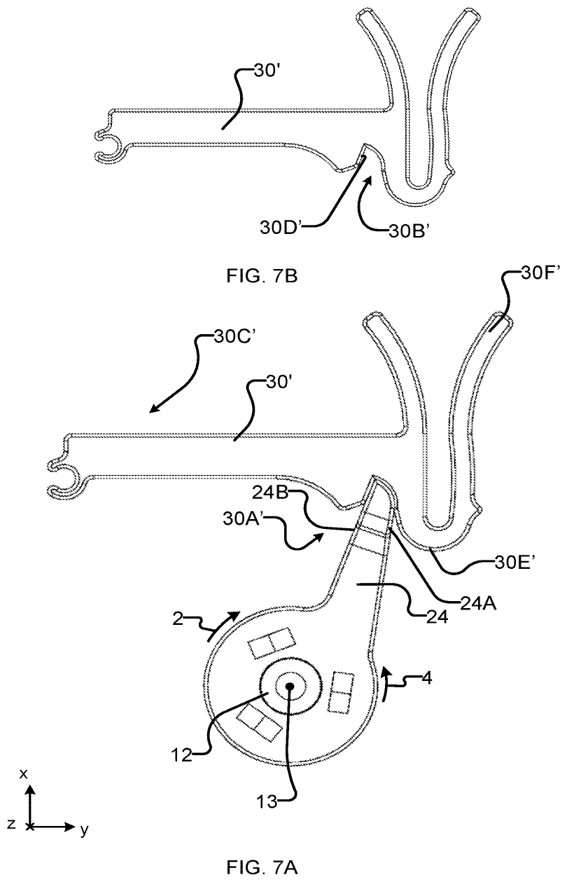

FIG. 7A shows a catch 30′ engaging latch 24 according to an exemplary embodiment of the invention. Catch 30′ may replace catch 30. FIG. 7B shows a catch 30′ according to an exemplary embodiment of the invention. Unlike catch 30 which is manually inserted in and removed from aperture 14A in housing 14 to engage and disengage latch 24, catch 30′ is configured to automatically engage latch 24 by resilient and restorative deformation, similar to catch 26. A first portion 30A′ of catch 30′ defines a slot 30B′ that is at least partially complementary in shape to latch 24 such that at least a portion of latch 24 may be received within slot 30B′. When latch 24 is engaged within slot 30B′ (as shown in FIG. 7A), abutment of latch 24 with an inner surface 30D′ of slot 30B′ prevents first direction 2 and second direction 4 rotation of shaft 12 thereby fixing shaft 12 in first position 12-1. A second portion 30C′ of catch 30 may be attached to housing 14 to fix catch 30′ relative to housing 14.

Latch 24 and catch 30 may be engaged as follows. As shaft 12 rotates in second direction 4, latch 24 also rotates until a trailing edge 24B of latch 24 contacts a beveled edge 30E′ of catch 30′. If shaft 12 continues to rotate (e.g. by application of an external force to shaft 12), contact of trailing edge 24B and beveled edge 30E′ causes resilient deformation of catch 30′ (and/or latch 24) to allow latch 24 to rotate into slot 30B′. After latch 24 has rotated into slot 30B′, catch 30′ (and/or latch 24) begins to restoratively deform or snap back, at least partially, toward its non-deformed shape (e.g. due to the restorative deformation forces associated with the resilient deformation of catch 30′). When catch 30′ has restoratively deformed or snapped back (at least partially) toward its non-deformed shape, abutment of latch 24 with an inner surface 30D′ of slot 30B′ prevents first direction 2 and second direction 4 rotation of shaft 12 thereby fixing shaft 12 in first position 12-1. In some embodiments, catch 30′ may be pivotably attached to housing 14 to allow rotation of catch 30′ about a z-direction axis to facilitate engagement and disengagement of latch 24 and slot 30B′.

In some embodiments, engagement of latch 24 and catch 30′ is releasable by applying external force (e.g. with a finger, an actuator, etc.) to a trigger 30F′. Trigger 30F′ may be attached to catch 30′ such that by applying external force to trigger 30F′, catch 30′ is resiliently deformed and/or pivoted about a z-direction axis to thereby allow latch 24 to rotate out of slot 30B′ in first direction 2.

While first position 12-1 is depicted herein as being a “12 o'clock” position such that latch 24 points in the x-direction, this is not mandatory. First position 12-1 may be any desired angular position of shaft 12 (and latch 24). It may be desirable to adjust the angular position of first position 12-1. To allow for different first positions 12-1, different catches 30, 30′ having slots 30D, 30D′ oriented at different angles may be provided. For example, a first catch 30 may be provided which sets first position 12-1 at an “11 o'clock position”, a second catch 30 may be provided which sets first position 12-1 at an “12 o'clock position” (as shown) and a third catch 30 may be provided which sets first position 12-1 at a “1 o'clock position”. Of course, catches 30 may be provided to set first position 12-1 at other orientations.

The angular difference between the first position 12-1 and the second position 12-2 may be any desired amount. In some embodiments, the angular difference between the first position 12-1 and the second position 12-2 is between approximately 10° and 180°. In some embodiments, the angular difference between the first position 12-1 and the second position 12-2 is between approximately 5° and 70°. In some embodiments, the angular difference between the first position 12-1 and the engaged position is between approximately 10° and 25°.

To facilitate rotation of shaft 12 in first and second direction 2, 4 about longitudinal axis 13, a handle 32 may be fixed to shaft 12. A user may apply force to handle 32 to rotate shaft 12 in first and second direction 2, 4 about longitudinal axis 13, as desired. To prevent a user from rotating shaft 12 further than intended, one or more stops 32A may be provided to contact handle 32 and thereby prevent further rotation of shaft 12 when a maximum intended rotation of shaft 12 is achieved. Stops 32A may coincide with first position 12-1 and/or second position 12-2, but this is not mandatory. Stops 32A may allow for rotation of shaft past first position 12-1 and/or second position 12-2.

In some embodiments, measuring apparatus 10 may comprise one or more force sensors 38 to measure a normal force (e.g. in the z-direction) applied to skin sample 5 by shaft 12 (or probe 20). Force sensors 38 may comprise any suitable force or strain sensors. Force sensors 38 may be attached to shaft 12, probe 20, or any other suitable portion of measuring apparatus 10. Force sensors 38 may facilitate ensuring that a consistent normal force is applied to skin sample 5 when measuring apparatus 10 is employed. Since, it is currently understood that the amount of normal force applied to skin sample 5 by shaft 12 (or probe 20) may effect the behaviour of skin sample 5 when skin measuring apparatus 10 is employed, it may be desirable to control, reduce or even avoid normal force applied to skin sample 5 during a single measurement and/or across multiple measurements with skin measuring apparatus 10.

In some embodiments, measuring apparatus 10 comprises one or more motors or actuators to cause rotation of shaft 12 from first position 12-1 to second position 12-2. Such motors or actuators may comprise hydraulic, pneumatic, electromechanical or electromagnetic motors or actuators such as, for example, an electric motor, a stepper motor, a servomotor, etc. For example, in some embodiments, shaft 12 is magnetic (or comprises magnetic portions) and may be rotated by driving current through one or more coils adjacent to shaft 12 to produce a magnetic field which causes shaft 12 to rotate, as desired (e.g. from first position 12-1 to second position 12-2). Where a motor or actuator is employed to rotate shaft 12 from first position 12-1 to second position 12-2, one or more features discussed herein may be omitted (e.g. latching mechanism 25, handle 32, catch 30, etc.) from measuring apparatus 10 since the motor or actuator or coils may be employed to control the angular position of shaft 12 about its longitudinal axis 13.

Skin measuring apparatus 10 may comprise one or more controllers 34 connected to control position sensor 18 and/or force sensor 22 and/or force sensor 38 (and/or optional actuators and/or motors). In some embodiments, controllers 34 may be replaced by the controller of another device (e.g. a cloud computing system, a smartphone, a tablet, a computer or the). Skin measuring apparatus 10 may comprise memory 36 for storing data obtained by sensor 18 and/or force sensor 22. Data obtained by sensor 18 and/or force sensor 22 may be stored in memory 36 until the data is manually caused to download to another device (e.g. a smartphone, a tablet or a computer) and/or data stored in memory 36 may be automatically uploaded (by wired connection or wirelessly) to another device or a network (e.g. a cloud computing system). In some embodiments, skin measuring apparatus 10 has no internal memory and is instead connected to another device (e.g. a smartphone, a tablet a computer, or the like) and data obtained by sensor 18 and/or force sensor 22 is stored on the device rather than in a memory of skin measuring apparatus 10.

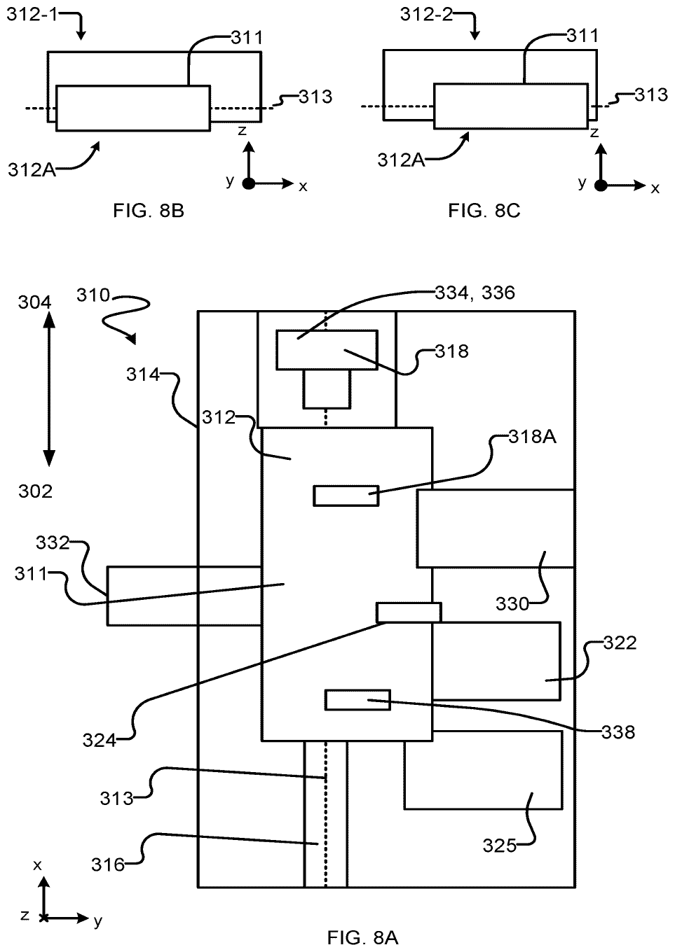

While mover 11 of skin measuring apparatus 10 is described herein as comprising a shaft 12 that is rotatable to cause deformation of skin sample 5, this is not mandatory. Instead, mover 11 may be linearly translatable to cause deformation of skin sample 5. For example, FIG. 8A depicts a skin measuring apparatus 310 having a linearly translatable mover 311 according to an exemplary embodiment of the invention. FIGS. 8B and 8C depict mover 311 of skin measuring apparatus 310 in first and second positions 312-1, 312-2.

Skin measuring apparatus 310 comprises a mover 311 that is linearly translatable in the x-direction to deform a skin sample 5 that is releasably attachable to mover 311. Skin measuring apparatus 310 is substantially similar to skin measuring apparatus 10 except as described herein.

Skin measuring apparatus 310 comprises a mover 311 releasably attachable to a skin sample 5. Mover 311 comprises a body 312 translatably attached to a housing 314 such that body 312 remains free to translate along x-direction axis 313 relative to housing 314 (e.g. housing 314 may remain stationary while body 312 translates along axis 313) in a first direction 302 and a second direction 304 (opposite first direction 302). Body 312 may be attached to housing 314 by one or more rails, tracks or the like to allow body 312 to translate along axis 313 relative to housing 314. For example, in the illustrated embodiment, body 312 is slidably attached to a first track 316 to allow body 312 to translate relative to housing 314. Body 312 may otherwise be fixed relative to housing 314 (e.g. in the y, and z directions and/or about the x, y and z-direction axes of body 312).

A first end 312A of body 312 may be releasably attachable to skin sample 5. Body 312 may be releasably attachable to skin sample 5 directly or indirectly by any suitable fastening means such as, for example, a suitable adhesive (e.g. doubled sided tape, cyanoacrylate adhesive, silicone adhesive, rosin adhesive, etc.). In this way, body 312 may be removed from skin sample as desired by releasing the adhesive (e.g. with suitable force, solvents, etc.).

Skin measuring apparatus 310 may comprise a position sensor 318 for determining a position of body 312 along axis 313. Position sensor 318 may comprise any suitable sensor for determining a position of body 312 along axis 313. Position sensor 318 may comprise a linear position sensor such as, for example, a capacitance sensor, an eddy current sensor, a photoelectric sensor, an ultrasonic sensor, a linear potentiometer, etc. In some embodiments, position sensor 318 is fixed to housing 314 and position sensor 318 tracks a target 318A attached to body 312. For example, in some embodiments, position sensor 318 comprises a magneto-resistive sensor or Hall Effect sensor fixed to housing 314 that tracks the position of one or more magnetic targets 318A attached to body 312. Position sensor 318 may comprise a camera (e.g. a video camera) for optical analysis of the linear position of body 312 or a target or marking on body 312.

Skin measuring apparatus 310 may comprise a force sensor 322 for measuring a force applied to body 312 by skin 5 (e.g. in the x-direction). Force sensor 322 may comprise any suitable sensor for measuring a force applied to body 312 (e.g. by skin sample 5). Force sensor 322 may measure the force applied to body 312 (e.g. by skin sample 5) directly or indirectly.

In practice, it may be desirable to control the amount of translation of body 312 along axis 313 when skin measuring apparatus 310 is employed to measure one or more characteristics of skin sample 5. Skin measuring apparatus 310 may comprise one or more features to facilitate achieving a desired amount of translation of body 312. In some embodiments, skin measuring apparatus 310 comprises a mechanism to control an amount of translation of body 312. In some embodiments, the amount of translation of body 312 is controlled by setting a separation distance between first and second positions 312-1, 312-2.

In some embodiments, skin measuring apparatus 310 comprises one or more features to facilitate setting skin measuring apparatus at a first position 312-1 (e.g. a starting position) and/or one or more features to facilitate setting skin measuring apparatus at a second position 312-2 (e.g. a taut position). Such features may comprise, for example, one or more of visual indicators, a detent mechanism, a mechanical latching mechanism, an electro-mechanical mechanism, an electro-magnetic mechanism, a magnetic mechanism, etc.

In some embodiments, skin measuring apparatus 310 comprises a selectively releasable latching mechanism 325. Latching mechanism 325 may comprise any suitable latching mechanism. Latching mechanism 325 may comprise a spring-loaded catch, a manual catch or latch, a ratcheting mechanism, a magnetic mechanism, an electromagnetic mechanism, an electromechanical mechanism, etc. Latching mechanism 325 may facilitate achieving a desired amount of translation of body 312. For example, latching mechanism may facilitate achieving second position 312-2 when translating body 312 in first direction 302. By engaging latching mechanism 325, latching mechanism 325 may prevent undesired translation of body 312 in second direction 304 from second position 312-2 until latching mechanism 325 is disengaged.

In some embodiments, a force applied to body 312 by skin sample 5 is determined indirectly by measuring a force at latching mechanism 325.

In some embodiments, a catch 330 is provided to engage latch 324 to thereby set body 312 at a first position 312-1. In some embodiments, catch 330 is manually engaged to hold body 312 at first position 312-1. In some embodiments, catch 330 is automatically engageable to hold body 312 at first position 312-1. In some embodiments, catch 330 is similar to catch 326 except in that it holds latch 324 at first position 312-1 rather than at second position 312-2. In some embodiments, catch 330 engages latch 324 to prevent first direction 302 translation of body 312. In some embodiments, catch 330 engages latch 324 to prevent first direction 302 translation and/or second direction 304 translation of body 312.

In some embodiments, measuring apparatus 310 may comprise one or more force sensors 338 to measure a normal force (e.g. in the z-direction) applied to skin sample 5 by body 312 (or a probe attached to body 312). Force sensors 338 may comprise any suitable force or strain sensors. Force sensors 338 may be attached to body 312, a probe attached to body 312 or any other suitable portion of measuring apparatus 310. Force sensors 338 may facilitate ensuring that a consistent normal force is applied to skin sample 5 when measuring apparatus 310 is employed. Since, it is currently understood that the amount of normal force applied to skin sample 5 by body 312 may effect the behaviour of skin sample 5 when skin measuring apparatus 310 is employed, it may be desirable to control, reduce or even avoid normal force applied to skin sample 5 during a single measurement and/or across multiple measurements with skin measuring apparatus 310.

In some embodiments, measuring apparatus 310 comprises one or more motors or actuators attached to body 312 to cause translation of body 312 along axis 313 from first position 312-1 to second position 312-2. Such motors or actuators may comprise hydraulic, pneumatic, electromechanical or electromagnetic motors or actuators such as, for example, an electric motor, a stepper motor, a servomotor, etc. For example, in some embodiments, body 312 is magnetic (or comprises magnetic portions) and current may be rotated by driving current through one or more coils adjacent to body 312 to produce a magnetic field which causes body 312 to translate, as desired (e.g. from first position 312-1 to second position 312-2). Where a motor or actuator is employed to translate body 312 from first position 312-1 to second position 312-2, one or more features discussed herein may be omitted (e.g. latching mechanism 325, handle 332, etc.) from measuring apparatus 310 since the motor or actuator may be employed to control the position of body 312 along axis 313.

Like skin measuring apparatus 10, skin measuring apparatus 310 may comprise one or more controllers 334 and/or memory 336 connected to control position sensor 318 and/or force sensor 322 (and/or optional actuators and/or motors).

In some embodiments, skin measuring apparatus 10 and/or skin measuring apparatus 310 may comprise one or more sensors, cameras or the like to monitor one or more markers placed on skin sample 5 at locations spaced apart from mover 11, 311. By tracking such markers before, during and/or after the mover is moved and skin sample 5 is deformed, further information as to how skin sample 5 is deformed by movement of the mover may be obtained.

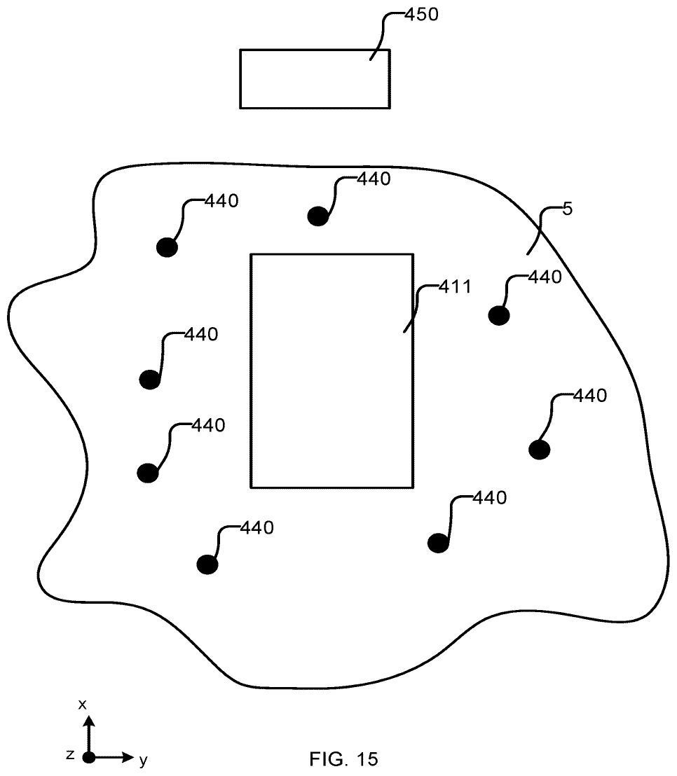

FIG. 15 depicts a mover 411 (which could comprise mover 11, mover 311 or any other mover) placed on a skin sample 5. A plurality of markers 440 are placed on skin sample 5 at locations spaced apart from mover 411. A sensor 450 is provided to monitor a position of each marker 440 before, during and/or after movement (e.g. rotation or translation) of mover 411.

Markers 440 may comprise any suitable markers. Markers 440 may comprise stickers, paint, ink or the like that are tracked optically by sensor 450. Markers 440 may have any suitable shape. Markers may comprise magnets, metallic markers, near-field communication chips or circuits (e.g. passive or active), transmitters, or the like that may be electromagnetically tracked or tracked with radio-wave based tracking, etc. Markers 440 may be placed equidistantly from mover 411 or at different distances from 411. Markers 440 may be spread around mover 411 in a set pattern (e.g. symmetrically) or in a random or pseudo-random manner.

Sensor 450 may comprise any suitable sensor such as, for example, an optical sensor, a camera (still or video), an electromagnetic sensor, a radio sensor, etc. Sensor 450 may track, for example, the position of one or more of markers 440 and/or the shape of one or more of markers 440 (and how that shape is deformed during and after movement of mover 411). Sensor 450 may directly measure the position and/or deformation of markers 440 or may provide data (e.g. images) that can be analyzed subsequently to determine the position and/or deformation of markers 440. Sensor 450 may comprise part of skin measuring apparatus 10, 310 or may be provided separately.

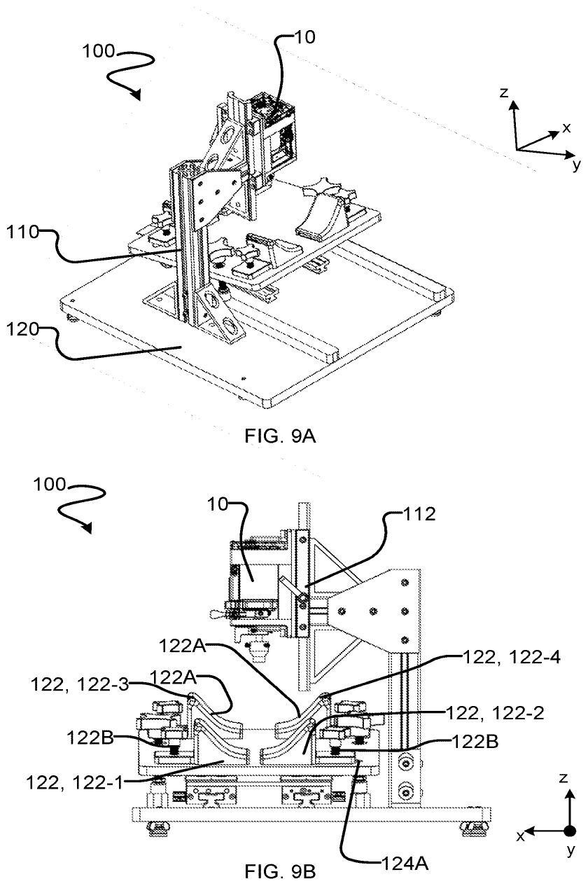

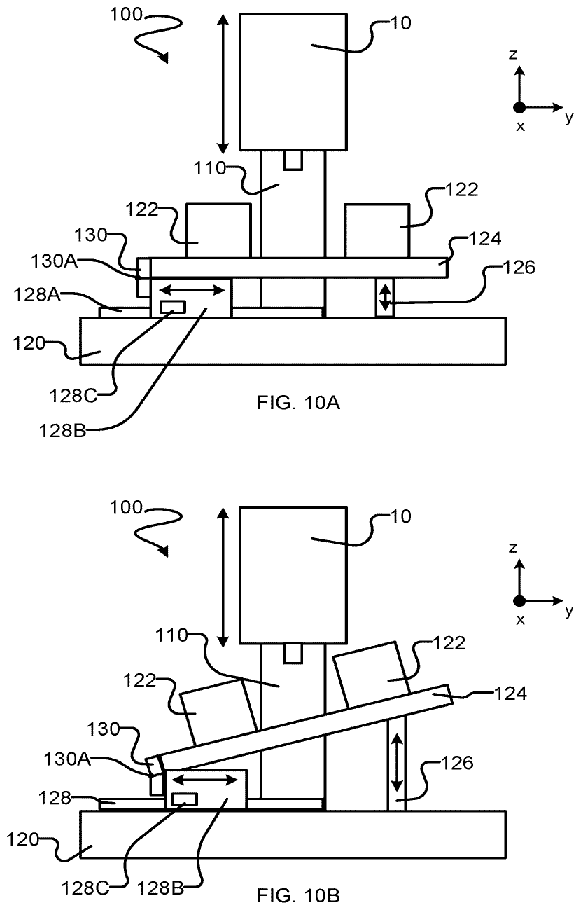

In practice, it may be desirable to reduce or even prevent relative movement between housing 14 of skin measuring apparatus 10 (or housing 314 of skin measuring apparatus 310) and the body part 7 on which skin sample 5 is located. For example, if body part 7 is an arm, it may be desirable to reduce relative movement between the arm and housing 14 of skin measuring apparatus 10, 310. FIGS. 9A and 9B show a stand 100 for supporting a skin measuring apparatus (such as skin measuring apparatus 10 or skin measuring apparatus 310) according to an exemplary embodiment of the invention. FIGS. 10A and 10B are schematic depictions of stand 100 to better illustrate some of the features of stand 100. Stand 100 may be employed to reduce or even prevent relative movement between housing 14 (or housing 314) and body part 7 (e.g. an arm, leg, etc.) on which skin sample 5 is located. While stand 100 may be employed to hold arm or leg, stand 100 could be suitably modified to hold another body part 7 such as a head to allow testing of a skin sample 5 on a user's face.

Stand 100 comprises an arm 110 supported by a base 120. Housing 14, 314 of skin measuring apparatus 10, 310 may be releasably attachable to arm 110 (directly or indirectly) to thereby hold skin measuring apparatus 10, 310 in a fixed position relative to base 120. One or more holders 122 for receiving body part 7 may be attached (directly or indirectly) to base 120 to provide a cradle, clamp or the like to hold body part 7 steady relative to base 120 (and skin measuring apparatus 10, 310). The position and orientation of skin measuring apparatus 10, 310 relative to base 120 may be adjustable to accommodate body parts 7 of different sizes and shapes. The position and orientation of holders 122 relative to base 120 may be adjustable to accommodate body parts 7 of different sizes and shapes.

In some embodiments, a releasable clamping mechanism 112 attaches housing 14, 314 of skin measuring apparatus 10, 310 to arm 110. When clamping mechanism 112 is released, skin measuring apparatus 10, 310 may be moved (e.g. in the z-direction and/or about an x-direction axis) relative to arm 110. When skin measuring apparatus 10, 310 is at a desired position (e.g. in the z-direction and/or about an x-direction axis), clamping mechanism 112 can be engaged to thereby fix skin measuring apparatus 10 relative to arm 110 (e.g. in and/or about x, y and z-directions axes). Any suitable clamping mechanism 112 may be employed. In this way, stand 100 may accommodate body parts 7 of different shapes and/or sizes while maintaining a desired z-direction distance between skin measuring apparatus 10, 310 and skin sample 5 of body part 7.

In some embodiments, base 120 supports a platform 124 on which holders 122 are mounted. The position and/orientation of platform 124 relative to base 120 may be adjustable to accommodate body parts 7 of different shapes and/or sizes. For example, in some embodiments, a mechanism is provided to selectively allow for x or y-direction movement of platform 124 relative to base 120, as desired. In some embodiments, a mechanism is provided to selectively allow for changes in orientation (e.g. about one or more of x, y and z direction axes) of platform 124 relative to base 120, as desired.

In some embodiments, platform 124 is attached to base 120 by one or more rails 128A that allow x and/or y-direction movement of platform 124 relative to base 120. In the illustrated embodiment, rails 128A are fixed to base 120 while platform 124 is attached to a slider 128B that can slide along rail(s) 128A in the y-direction while preventing or reducing x and z-direction movement between rail(s) 128A and slider 128B. Various elements may be provided to reduce friction between rail(s) 128A and slider 128B such as, but not limited to, wheels, bearings, cartridge bearings, low friction materials, etc.

When it is desired to prevent y-direction movement of platform 124 relative to base, a selectively releasable clamp may be engaged. In the illustrated embodiments, a releasable clamp 128C is provided to fix slider 128B to rail(s) 128A to thereby prevent y-direction movement of platform 124 relative to base 120. Clamp 128C may comprise a set screw style clamp, a quick release lever style clamp, or any other suitable style of clamp to prevent relative movement between slider 128B and rail(s) 182A which thereby prevents y-direction movement of platform 124 relative to base 120.

In some embodiments, platform 124 is pivotably (e.g. hingably) attached to base 120 (or to slider 128B) to allow platform 124 to rotate about an x-direction axis. For example, in the illustrated embodiment, a hinge 130 attaches platform 124 to slider 128B. Hinge 130 allows platform 124 to rotate about an x-direction axis 130A, as shown in FIGS. 10A and 10B. Rotating platform 124 about x-direction axis 130A may facilitate accommodating body parts 7 that are tapered such as, for example, a forearm or a thigh. Specifically, by rotating platform 124 about an x-direction axis, it may be possible to align skin measuring apparatus 10, 310 (e.g. probe 20 of skin measuring apparatus 10) with skin sample 5 such that a surface of skin sample 5 is arranged substantially parallel to a surface of skin measuring apparatus 10, 310 (e.g. surface 20A of probe 20 of skin measuring apparatus 10) and/or such that a surface of skin sample 5 is substantially orthogonal to longitudinal axis 13 of shaft 12 of measuring apparatus 10.

Platform 124 may also be supported above base 120 by one or more adjustable supports 126. A first end of each adjustable support 126 may be fixed to platform 124 while a second end of each adjustable support 126 is attached to or rests on base 120 to thereby support platform 124. Adjustable supports 126 may be adjustable in z-direction length to accommodate rotation of platform 124 about an x-direction axis (e.g. x-direction axis 130A) as shown for example, in FIG. 10B. For example, when platform 124 is rotated in a counter-clockwise direction about x-direction axis 130A to go from the FIG. 10A position to the FIG. 10B position, the effective z-direction length of adjustable support 126 can be increased to thereby support platform 124 on base 120. Similarly, when platform 124 is rotated in a clockwise direction about x-direction axis 130A to go back from the FIG. 10B position to the FIG. 10A position, the effective z-direction length of adjustable support 126 can be decreased to thereby support platform 124 on base 120. In some embodiments, adjustable supports 126 are threadedly attached to platform 124 or base 120 such that by rotating a handle attached to supports 126, the effective z-direction length of adjustable supports is changed.

One or more holders 122 may be attached to platform 124 to receive body part 7. Holders 122 may prevent or reduce undesirable movement of body part 7 relative to platform 124 and base 120. In some embodiments, each holder comprises a concave surface 122A for receiving a portion of body part 7, as shown in FIG. 9B. Holders 122 may comprise a first pair of holders 122-1 and 122-2 opposing each other in the x-direction. Holders 122 may comprise a second pair of holders 122-3 and 122-4 opposing each other in the x-direction and spaced apart from holders 122-1, 122-2 in the y-direction. Each pair of holders 122 may define a cradle for receiving body part 7 between concave surfaces 122A.

The x-direction width of the cradle defined between concave surfaces 122A of opposing holders 122 may be adjusted by adjusting the spacing between opposing holders 122. In this way, holders 122 may allow for body parts 7 of various sizes (e.g. a forearm vs. a thigh). In some embodiments, each holder 122 is attached to platform 124 by a fastener (e.g. bolt 122B) received in a slot 124A defined by platform 124. Slots 124A may each extend generally in the x-direction such that sliding the fastener within slot 124A in the x-direction allows opposing holders 122 to be moved relative to one another in the x-direction. When a desired position of a holder 122 is achieved, the fastener may be tightened (e.g. a nut may be tightened on bolt 122B) to fix holder 122 to platform 124.

In some embodiments, body part 7 is merely allowed to rest in the cradle defined between opposing holders 122 (e.g. holders 122-1, 122-2) when skin measuring apparatus 10, 310 is in use. In some embodiments, further securing elements are provided to reduce movement of body part 7 relative to platform 124. For example, in some embodiments, straps, clamps or the like are provide to hold body part 7 in place in the cradle defined between opposing holders 122 (e.g. in the cradle defined between holders 122-1, 122-2 and/or in the cradle defined between holders 122-3, 122-4).

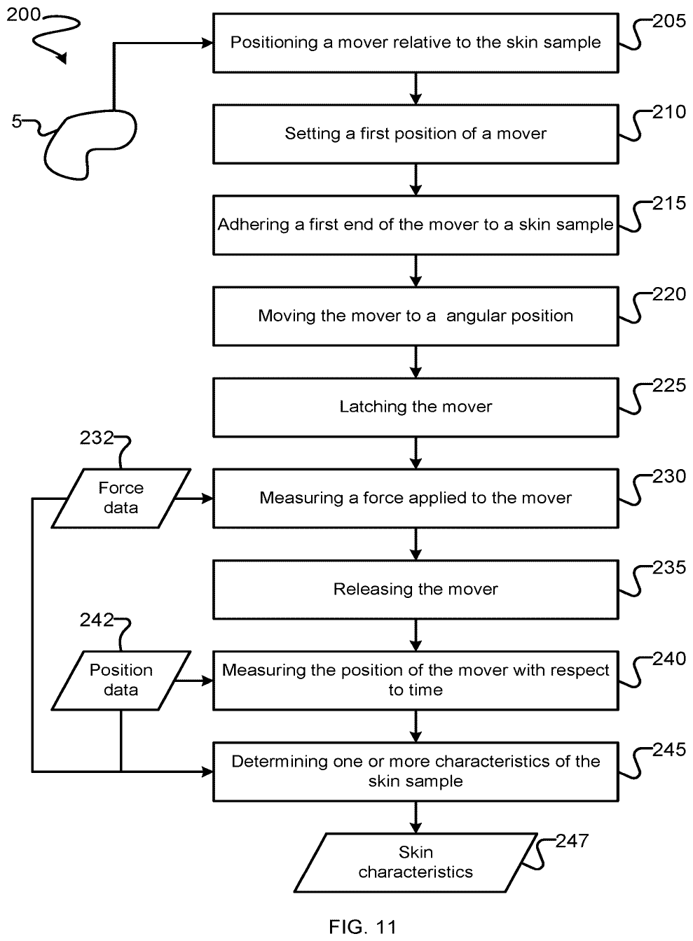

FIG. 11 depicts a method 200 for measuring one or more characteristics of a skin sample 5 according to an exemplary embodiment of the invention. For convenience, method 200 may be described herein as employing skin measuring apparatus 10 and/or skin measuring apparatus 310 but it should be understood that method 200 may employ skin measuring apparatus 10, skin measuring apparatus 310, stand 100 and/or any suitable skin measuring apparatus and/or stand.

Method 200 may start at block 205 with positioning a mover 211 relative to a skin sample 5. Mover 211 may comprise any suitable mover. Mover 211 may comprise a rotational mover 211′ (e.g. like mover 11) or a linear mover 211″ (e.g. like mover 311). In some embodiments, mover 211 comprises shaft 12 of skin measuring apparatus 10 or body 312 of skin measuring apparatus 310, but this is not mandatory. In some embodiments, positioning mover 211 relative to skin sample 5 comprises placing a body part 7 in stand 100 and adjusting one or more of the z-direction height of skin measuring apparatus 10 (or skin measuring apparatus 310), the position and/or orientation of platform 124 and the position and/or orientation of holders 122. In some embodiments, block 205 merely comprises bringing mover 211 close to skin sample 5.

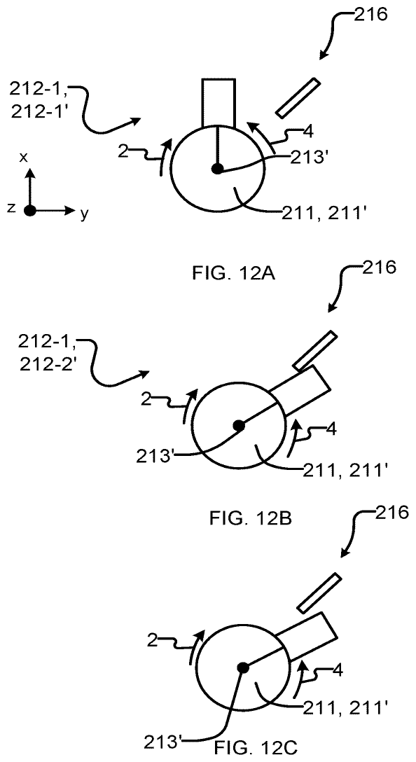

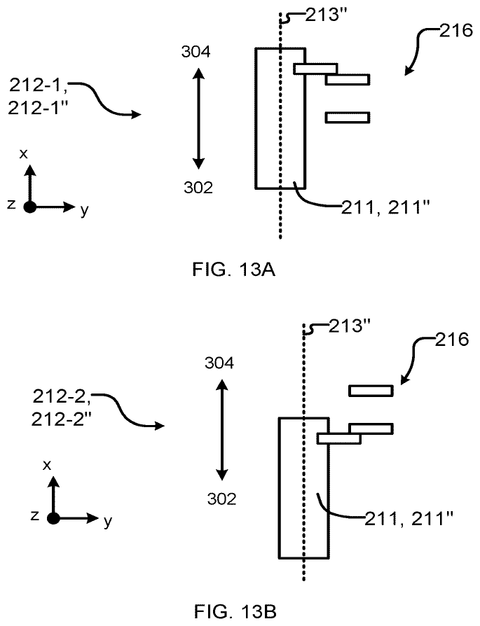

Method 200 may start at block 210 (i.e. block 210 may happen before or after block 205) with setting a position of mover 211 to a first position 212-1. First position 212-1 may comprise a first position 212-1′ as shown in FIG. 12A. First position 212-1′ may be the same as first position 12-1, as shown in FIGS. 5A and 6B. For simplicity, first position 212-1′ of mover 211′ is shown as mover 211′ being oriented at a “12 o'clock” position, but this is not mandatory. It should be understood that first position 212-1′ could comprise any suitable angular orientation of mover 211′. First position 212-1 may comprise a first position 212-1″, as shown in FIG. 13A. First position 212-1″ may be the same as first position 312-1, as shown in FIG. 8B.

In some embodiments, to achieve first position 212-1, a human operator may adjust mover 211 based on their visual perception of the position of mover 211 to achieve as close to a desired first position 212-1 as possible without any aids (i.e. by “eyeballing” the position of mover 211). In other embodiments, one or more mechanical, electro-mechanical, magnetic and/or visual, aids may be employed to more accurately achieve a desired first position 212-1. Where method 200 employ skin measuring apparatus 10, catch 30 (or catch 30′) may be employed to accurately achieve a desired first position 212-1′, as shown in FIG. 6B or FIG. 7B. Where method 200 employ skin measuring apparatus 310, catch 330 may be employed to accurately achieve a desired first position 212-1″. In some embodiments, mover 211 is driven by an actuator or motor (e.g. a rotary actuator such as a servomotor, stepper motor, etc.) to precisely control a position or orientation of mover 211. In some embodiments, one or more markings may be placed on mover 211 to assist in achieving first position 212-1.

Once first position 212-1 of mover 211 is achieved, mover 211 may be held at that orientation by friction, manually (e.g. by a human operator), automatically (e.g. by a motor or actuator), mechanically (e.g. by catch 30, catch 30′, catch 330 or the like), etc.

At block 215, a first end of mover 211 is adhered to skin sample 5. In some embodiments, block 215 occurs after blocks 205 and 210. In some embodiments, block 215 occurs after block 205 but before block 210. Mover 211 may be adhered to skin sample 5 using any suitable mechanism or adhesive. For example, where skin measuring apparatus 10 is used for method 200, probe surface 20A may be adhered to skin sample 5 with a suitable adhesive (e.g. an adhesive sticker). The adhesion between mover 211 and skin sample 5 should be sufficiently strong to prevent or reduce slipping between mover 211 and skin sample 5 such that skin sample 5 is deformed as mover 211 is moved (e.g. rotated or translated) at later blocks of method 200 (e.g. block 220 as discussed further herein).

In some embodiments, method 200 comprises determining a normal force applied to skin sample 5 by mover 211 (e.g. in the z-direction) and/or adjusting a z-direction position of mover 211 to achieve a desired normal force applied to skin sample 5 by mover 211. This may occur before block 220, before block 225, before block 230 or at any desired time during method 200. In some embodiments, one or more force sensors (e.g. force sensor 38 or force sensor 338) may be employed to determine and facilitate setting a desired normal force applied to skin sample 5 by mover 211. Setting a desired normal force may facilitate achieving accurate, reliable, consistent and/or repeatable results when employing method 200. It may be desirable that the normal force is relatively close to zero to prevent indentation or stretching of or otherwise affecting skin sample 5 at first position 212-1.

After block 215, method 200 continues to block 220. At block 220, mover 211 is moved from first position 212-1 to second position 212-2. For example, where a rotational mover 211 is employed (e.g. skin measuring apparatus 10 is employed), mover 211 is rotated about its longitudinal axis 213′ in first direction 2 until mover 211 reaches a second position 212-2′ as shown in FIG. 12B. Second position 212-2 may be the same as second position 12-2 as shown in FIG. 5C.

An angular difference between first position 212-1′ and second position 212-2′ may be between approximately 10° and 180°. In some embodiments, the angular difference between first position 212-1′ and second position 212-2′ is between approximately 5° and 70°. In some embodiments, the angular difference between first position 212-1′ and second position 212-2′ is between approximately 10° and 30°.

As another example, where a linear mover 211 is employed (e.g. skin measuring apparatus 310 is employed), mover 211 is translated along axis 213″ in first direction 302 until mover 211 reaches second position 212-2″ as shown in FIG. 13B. Second position 212-2 may be the same as second position 312-2 as shown in FIG. 8C.

A distance between first position 212-1″ and second position 212-2″ may be between approximately 5 mm and 50 mm. In some embodiments, the distance between first position 212-1″ and second position 212-2″ may be between approximately 10 mm and 30 mm.

At block 220, mover 211 may be moved to second position 212-1 using any suitable technique or mechanism. For example, mover 211 may be moved manually (e.g. by a human operator) or automatically (e.g. by an actuator or motor such as, but not limited to, an electric motor, a stepper motor, a servomotor, an electromagnetic motor, etc.). Where skin measuring apparatus 10 is employed, a human operator may apply force to handle 32 to rotate shaft 12 of skin measuring apparatus 10 from first position 212-1′ to second position 212-2.′

In some embodiments, to achieve second position 212-2, a human operator adjusts mover 211 based on their visual perception of the position of mover 211 to achieve as close to a desired second position 212-2 as possible without any aids (i.e. by “eyeballing” the position of mover 211). In other embodiments, one or more visual, mechanical electro-mechanical or magnetic aids may be employed to more accurately achieve a desired second position 212-2. In some embodiments, mover 211 is driven by an actuator or motor (e.g. a servomotor, stepper motor, or the like) to precisely control a position or orientation of mover 211. In some embodiments, mover 211 has one or more markings to assist in achieving a desired second position 212-2.

After block 220 (and before block 235), mover 211 may be held at second position 212-2. Mover 211 may be held at second position 212-2 manually (e.g. by a human operator), by an actuator or motor and/or by a catch mechanism 216. Where catch mechanism 216 is employed, method 200 comprises an optional block 225 where mover 211 is latched by catch mechanism 216 (e.g. such as catch mechanism 25 of skin measuring apparatus 10 or catch mechanism 325 of skin measuring apparatus 310) so as to (1) accurately achieve second position 212-2 and/or (2) hold mover 211 at second position 212-2 until block 235. Catch mechanism 216 may comprise any suitable catch mechanism. In some, mover 211 may be moved (e.g. rotated or translated) slightly past second position 212-2 at block 220 (e.g. as shown in FIG. 12C) to allow or cause catch mechanism 216 to engage before catch mechanism 216 can hold mover 211 at second position 212-2. Once catch mechanism 216 is engaged, mover 211 may be returned to second position 212-2 where it is held in place by catch mechanism 216. Catch mechanism 216 may be engaged automatically when mover 211 reaches or passes second position 212-2 or may be engaged manually (e.g. by a human operator).

At block 230, a force applied to mover 211 by skin sample 5 is measured or determined to obtain force data 232. Movement of mover 211 from first position 212-1 to second position 212-2 may resiliently deform skin sample 5 due to the adhesion between skin sample 5 and mover 211. Such resilient deformation of skin sample 5 may create restorative deformation forces associated with a tendency of skin sample 5 to restoratively deform back to its non-deformed state. Due to the adhesion between skin sample 5 and mover 211, such restorative deformation forces (or a portion thereof) are applied to mover 211. This force may be measured or determined in any suitable manner at block 230. In some embodiments, a force sensor is placed between skin sample 5 and mover 211 to measure the force directly. In some embodiments, the force may be determined by measuring a force applied by mover 211 on latching mechanism 216. For example, where skin measuring apparatus 10 is employed for method 200, force data 232 is obtained by using force sensor 22 to measure a force applied by latch 24 on catch 26.

The force may be measured at one or more times during method 200. In some embodiments, the force is measured continuously throughout method 200 at set time intervals (e.g. every 1 second, every 1 millisecond, etc.). In some embodiments, force is measured when mover 211 is at second position 212-2. Force measured when mover 211 is at second position 212-2 may be the maximum force applied by skin sample 5 (unless mover 211 is moved past second position 212-2). In some embodiments, force data 232 comprises a torque applied to mover 211 by skin sample 5 determined at least in part based on a force measured when mover 211 is at second position 212-2.

At block 235, after mover 211 has reached second position 220 (and potentially after mover 211 is optionally latched and at least some of force data 232 is obtained), mover 211 may be released. Releasing mover 211 may comprise removing any outside forces or inhibitors that prevent mover 211 from freely moving (e.g. rotating about its longitudinal axis 213′ or moving along axis 213″) due to restorative deformation of skin sample 5. For example, releasing mover 211 may comprise disengaging catch mechanism 216 (e.g. by disengaging catch 26 where skin measuring apparatus 10 is employed), disengaging an actuator, releasing a human operator's grip on mover 211 or a handle of mover 211, etc.

When mover 211 is released at block 235, restorative deformation forces associated with the tendency of skin sample 5 to restore to its non-deformed state may cause mover 211 to move back toward first position 212-1 (e.g. rotate in second direction 4 toward first position 212-1′ or translate in second direction 304 toward first position 212-1″). In some cases, restorative deformation forces associated with the tendency of skin sample 5 to restore to its non-deformed state may cause mover 211 to move past first position 212-1 thereby causing secondary resilient deformation of skin sample 5. The magnitude of the secondary resilient deformation may be less than the initial deformation of skin sample 5 (when mover 211 was moved to second position 212-2) due to natural damping (e.g. energy dissipation) of skin sample 5. Restorative deformation forces associated with the secondary resilient deformation of skin sample 5 may cause mover 211 to move again toward second position 212-2 (e.g. in first direction 2 or first direction 302) thereby causing tertiary deformation of skin sample 5. The magnitude of the tertiary resilient deformation may be less than the secondary deformation of skin sample 5 again due to natural damping (e.g. energy dissipation) of skin sample 5. This oscillation of mover 211 in first and second directions 2, 4 (or first and second directions 302, 304) may continue until the energy in the system is dissipated due to the natural damping of skin sample 5.

The position (e.g. angular position about axis 213′ or linear position along axis 213″) of mover 211 after mover 211 is released at block 235 (and optionally starting at or before block 210 or block 220) may be measured at block 240 to obtain position data 242. In some embodiments, the position of mover 211 is measured at set time intervals (e.g. every 1 second, every 1 millisecond, etc.) starting at or before block 210, block 220, block 235 or at any other suitable time, as desired.

The position of mover 211 may be measured with any suitable technique or apparatus. For example, the position of mover 211 may be tracked by optical analysis (e.g. video or picture review), by a linear position sensor or by a rotary encoder such as, not limited to a mechanical absolute encoder, an optical absolute encoder, a magnetic absolute encoder, a capacitive absolute encoder, a geared multi-turn encoder, an incremental encoder, etc. Where skin measuring apparatus 10 is employed for method 200, the angular position of shaft 12 may be tracked by position sensor 18, as described further herein. Where skin measuring apparatus 310 is employed for method 200, the linear position of body 312 may be tracked by position sensor 318, as described further herein.

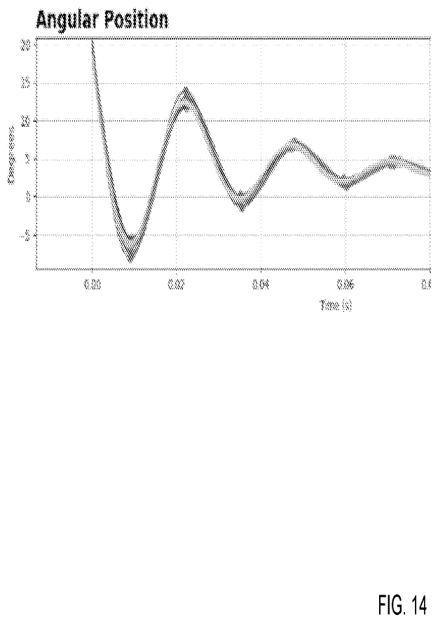

FIG. 14 depicts a plot of angular position (e.g. position data 242) measured in degrees from first position 212-1′ (e.g. first position 212-1′ is equal to 0°) with respect to time for a number of exemplary skin samples. In the case of the plot of FIG. 14, at a time of 0 seconds, the angular position of mover 211′ corresponded to a second position 212-2′ of 20° from first position 212-1′. As can be seen from FIG. 14, the angular position of mover 211′ exhibits oscillatory motion with an exponential decay in amplitude similar to what would be seen for an underdamped spring system.

In some embodiments, method 200 comprises an optional block of determining one or more characteristics 247 of skin sample 5 based at least in part on force data 232 and/or position data 242. In some embodiments, skin characteristics 247 comprise a parameterization of one or more of force data 232 and position data 242. For example, in some embodiments, skin characteristics 247 comprise one or more of the amplitude of the oscillation of mover 211, the frequency of the oscillation of mover 211 and the period of oscillation of mover 211 determined based at least in part on position data 242.

One or more of skin characteristics 247 obtained at block 245 may then be compared to known ranges of skin characteristics 247 (e.g. obtained through empirical testing) to determine how skin sample 5 compares.

Alternatively or additionally, a skin sample 5 may be tested by method 200 before and after one or more skin treatments (e.g. dieting, avoiding sun exposure and the use of various products and services, such as, cosmetics, botulinum, exfoliation, fillers, laser resurfacing, microdermabrasion, peels, retinol therapy, ultrasonic skin treatment, etc.) and one or more skin characteristics 247 obtained from method 200 before the skin treatment may be compared to one or more skin characteristics 247 obtained from method 200 after the skin treatment to determine the efficacy of the skin treatment.

### Interpretation

While a number of exemplary aspects and embodiments have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as are consistent with the broadest interpretation of the specification as a whole.

Where a component is referred to above, unless otherwise indicated, reference to that component (including a reference to a “means”) should be interpreted as including as equivalents of that component any component which performs the function of the described component (i.e. that is functionally equivalent), including components which are not structurally equivalent to the disclosed structure which performs the function in the illustrated exemplary embodiments of the invention.

Unless the context clearly requires otherwise, throughout the description and any accompanying claims (where present), the words “comprise,” “comprising,” and the like are to be construed in an inclusive sense, that is, in the sense of “including, but not limited to.” As used herein, the terms “connected,” “coupled,” or any variant thereof, means any connection or coupling, either direct or indirect, between two or more elements; the coupling or connection between the elements can be physical, logical, or a combination thereof. Additionally, the words “herein,” “above,” “below,” and words of similar import, shall refer to this document as a whole and not to any particular portions. Where the context permits, words using the singular or plural number may also include the plural or singular number respectively. The word “or,” in reference to a list of two or more items, covers all of the following interpretations of the word: any of the items in the list, all of the items in the list, and any combination of the items in the list.

Embodiments of the invention may be implemented using specifically designed hardware, configurable hardware, programmable data processors configured by the provision of software (which may optionally comprise “firmware”) capable of executing on the data processors, special purpose computers or data processors that are specifically programmed, configured, or constructed to perform one or more steps in a method and/or to provide the functionality as explained in detail herein and/or combinations of two or more of these. Examples of specifically designed hardware are: logic circuits, application-specific integrated circuits (“ASICs”), large scale integrated circuits (“LSIs”), very large scale integrated circuits (“VLSIs”), and the like. Examples of configurable hardware are: one or more programmable logic devices such as programmable array logic (“PALs”), programmable logic arrays (“PLAs”), and field programmable gate arrays (“FPGAs”). Examples of programmable data processors are: microprocessors, digital signal processors (“DSPs”), embedded processors, graphics processors, math co-processors, general purpose computers, server computers, cloud computers, mainframe computers, computer workstations, and the like. For example, one or more data processors in a control circuit for a device may implement methods and/or provide functionality as described herein by executing software instructions in a program memory accessible to the processors.

Software and other modules may reside on servers, workstations, personal computers, tablet computers, image data encoders, image data decoders, PDAs, media players, PIDs and other devices suitable for the purposes described herein. Those skilled in the relevant art will appreciate that aspects of the system can be practiced with other communications, data processing, or computer system configurations, including: Internet appliances, hand-held devices (including personal digital assistants (PDAs)), wearable computers, all manner of cellular or mobile phones, multi-processor systems, microprocessor-based or programmable consumer electronics, network PCs, mini-computers, mainframe computers, and the like.

While processes or blocks of some methods are presented herein in a given order, alternative examples may perform routines having steps, or employ systems having blocks, in a different order, and some processes or blocks may be deleted, moved, added, subdivided, combined, and/or modified to provide alternative or sub-combinations. Each of these processes or blocks may be implemented in a variety of different ways. Also, while processes or blocks are at times shown as being performed in series, these processes or blocks may instead be performed in parallel, or may be performed at different times. In addition, while elements are at times shown as being performed sequentially, they may instead be performed simultaneously or in different sequences. It is therefore intended that the following claims are interpreted to include all such variations as are within their intended scope.

Various features are described herein as being present in “some embodiments”. Such features are not mandatory and may not be present in all embodiments. Embodiments of the invention may include zero, any one or any combination of two or more of such features. This is limited only to the extent that certain ones of such features are incompatible with other ones of such features in the sense that it would be impossible for a person of ordinary skill in the art to construct a practical embodiment that combines such incompatible features. Consequently, the description that “some embodiments” possess feature A and “some embodiments” possess feature B should be interpreted as an express indication that the inventors also contemplate embodiments which combine features A and B (unless the description states otherwise or features A and B are fundamentally incompatible).

Specific examples of systems, methods and apparatus have been described herein for purposes of illustration. These are only examples. The technology provided herein can be applied to systems other than the example systems described above. Many alterations, modifications, additions, omissions, and permutations are possible within the practice of this invention. This invention includes variations on described embodiments that would be apparent to the skilled addressee, including variations obtained by: replacing features, elements and/or acts with equivalent features, elements and/or acts; mixing and matching of features, elements and/or acts from different embodiments; combining features, elements and/or acts from embodiments as described herein with features, elements and/or acts of other technology; and/or omitting combining features, elements and/or acts from described embodiments.

The invention comprises a number of non-limiting aspects. Non-limiting aspects of the invention provide:

1. A skin measuring apparatus for measuring skin characteristics, the skin measuring apparatus comprising:
a mover attached to a housing to allow the mover to move in a first direction and a second direction relative to the housing;
a position sensor attached to the housing for measuring a position of the mover as the mover moves relative the housing between first and second positions;
a force sensor for measuring a force applied to the mover;
a latch fixed to the mover;
a releasable catch attached to the housing, the releasable catch engageable with the latch to prevent movement of the mover in the second direction from the second position until the releasable catch is released;
wherein the first direction is opposite the second direction;
wherein a first end of the mover is releasably attachable to a skin sample.
   2. A skin measuring apparatus according to aspect 1 or any other aspect herein wherein a mover attached to a housing to allow the mover to rotate about a first axis in the first direction and the second direction relative to the housing.
   3. A skin measuring apparatus according to aspect 2 or any other aspect herein wherein the mover is rotatable in the first direction and/or the second direction by actuation of a rotary actuator or motor attached to the mover.
   4. A skin measuring apparatus according to any one of aspects 2 to 3 or any other aspect herein wherein the releasable catch is attached to a catch body, the catch body rotatable about the first axis.
   5. A skin measuring apparatus according to aspect 4 or any other aspect herein wherein the force sensor is fixed relative to the housing and a sensing portion of the force sensor is attached to the catch body to measure a torque about the first axis, the torque applied to the catch body by the latch.
   6. A skin measuring apparatus according to any one of aspects 2 to 5 or any other aspect herein wherein an angular difference between the first position and the second position is between approximately 10° and 180°.
   7. A skin measuring apparatus according to any one of aspects 2 to 5 or any other aspect herein wherein an angular difference between the first position and the second position is between approximately 5° and 70°.
   8. A skin measuring apparatus according to any one of aspects 2 to 5 or any other aspect herein wherein an angular difference between the first position and the second position is between approximately 10° and 25°.
   9. A skin measuring apparatus according to any one of aspects 2 to 8 or any other aspect herein wherein the mover is rotatably attached to the housing by one or more bearings.
   10. A skin measuring apparatus according to any one of aspects 2 to 8 or any other aspect herein wherein the mover is rotatably attached to the housing by one or more bushings.
   11. A skin measuring apparatus according to aspect 1 or any other aspect herein wherein mover attached to a housing to allow the mover to translate along a first axis in the first direction and the second direction relative to the housing.
   12. A skin measuring apparatus according to aspect 11 or any other aspect herein wherein the mover is translatable in the first direction and/or the second direction by actuation of an actuator or motor attached to the mover.
   13. A skin measuring apparatus according to any one of aspects 11 and 12 or any other aspect herein wherein a distance between the first position and the second position is between approximately 5 mm and 50 mm.
   14. A skin measuring apparatus according to any one of aspects 11 and 12 or any other aspect herein wherein a distance between the first position and the second position is between approximately 10 mm and 30 mm.

15. A skin measuring apparatus according to any one of aspects 11 to 14 or any other aspect herein wherein the mover is translatably attached to the housing by one or more rails.

16. A skin measuring apparatus according to any one of aspects 11 to 14 or any other aspect herein wherein the mover is translatably attached to the housing by one or more tracks.
17. A skin measuring apparatus according to any one of aspects 1 to 16 or any other aspect herein wherein a sensing portion of the force sensor is fixed to the mover.
18. A skin measuring apparatus according to any one of aspects 1 to 17 or any other aspect herein wherein the latch engages the releasable catch by resilient deformation of one or more of the latch and the catch.

19. A skin measuring apparatus according to any one of aspects 1 to 18 or any other aspect herein wherein the latch engages the releasable catch by restorative deformation of one or more of the latch and the catch.

20. A skin measuring apparatus according to any one of aspects 1 to 19 or any other aspect herein wherein the catch is resiliently deformable to thereby release the latch by application of an external force to a trigger.
21. A skin measuring apparatus according to aspect 20 or any other aspect herein wherein the trigger is accessible through an aperture defined by the housing.
22. A skin measuring apparatus according to any one of aspects 1 to 21 or any other aspect herein comprising a block releasably engageable with the latch to hold the mover at the first position by abutment of a first portion of the block with the housing and abutment of a second portion of the block with the latch.
23. A skin measuring apparatus according to aspect 22 or any other aspect herein wherein abutment of the second portion of the block with the latch comprises receiving the latch within a slot defined by the block.
24. A skin measuring apparatus according to any one of aspects 22 and 23 or any other aspect herein wherein abutment of the first portion of the block with the housing comprising inserting at least a portion of the block into an aperture defined by the housing.
25. A skin measuring apparatus according to any one of aspects 1 to 24 or any other aspect herein comprising a second releasable catch attached to the housing, the second releasable catch engageable with the latch to prevent movement of the mover from the first position until the second releasable catch is released
26. A skin measuring apparatus according to any one of aspects 1 to 25 or any other aspect herein comprising an adhesive for attaching the first end of the mover to the skin sample.
27. A skin measuring apparatus according to any one of aspects 1 to 26 or any other aspect herein wherein the position sensor comprises a Hall effect sensor fixed relative to the housing and a magnet fixed relative to the mover.
28. A skin measuring apparatus according to any one of aspects 1 and 27 or any other aspect herein wherein the releasable catch is spring-loaded.
29. A skin measuring apparatus according to any one of aspects 1 to 28 or any other aspect herein wherein the housing surrounds at least a portion of the mover.
30. A skin measuring apparatus according to any one of aspects 1 to 29 or any other aspect herein wherein the force sensor comprises a torque sensor.
31. A skin measuring apparatus according to any one of aspects 1 to 29 or any other aspect herein wherein the force sensor comprises a strain gauge.
32. A skin measuring apparatus according to any one of aspects to 1 to 31 or any other aspect herein comprising a stand, the stand comprising:
a base;
an arm extending in a z-direction from the base, wherein the housing is attached to the arm to support the housing above the base;
one or more holders supported by the base, the one or more holders defining a cradle to receive a body part of the skin sample.
   33. A skin measuring apparatus according to aspect 32 or any other aspect herein comprising a platform adjustably attached to the base, wherein the one or more holders are attached to the platform.
   34. A skin measuring apparatus according to aspect 33 or any other aspect herein wherein a position and/or orientation of the platform relative to the base is adjustable to accommodate a shape of the body part.
   35. A skin measuring apparatus according to aspect 33 or any other aspect herein wherein a y-direction position and/or an orientation about an x-direction axis of the platform relative to the base is adjustable to accommodate a shape of the body part, wherein the x, y and z-directions are mutually orthogonal.
   36. A skin measuring apparatus according to any one of aspects 34 and 35 or any other aspect herein wherein the platform is fixed to a slider attached a rail attached to the base to allow the platform to slide in the y-direction on the rail relative to the base.
   37. A skin measuring apparatus according to aspect 36 or any other aspect herein comprising a rail clamp selectively engageable to prevent relative movement between the platform and the rail.
   38. A skin measuring apparatus according to any one of aspects 36 to 37 or any other aspect herein wherein the platform is fixed to the slider by a hinge to allow rotation of the platform about an x-direction hinge axis.
   39. A skin measuring apparatus according to any one of aspects 32 to 38 or any other aspect herein wherein a z-direction distance between the housing and the base is adjustable by selectively releasing and locking a height clamp.
   40. A skin measuring apparatus according to any one of aspects 32 to 39 or any other aspect herein wherein positions and/or orientations of the one or more holders are adjustable to vary the size of the cradle.
   41. A method for measuring skin characteristics, the method comprising:
providing a mover attached to a housing to allow the mover to move in a first direction and a second direction relative to the housing;
setting a first position of the mover;
adhering a first end of the mover to a skin sample;
moving the mover in the first direction to a second position;
releasing the mover to allow the mover to move in the second direction;
measuring a force applied to the mover by the skin sample;
measuring the position of the mover while it moves in the first and second directions;
determining one or more skin characteristics based at least in part on the measured force and the measured angular position.
   42. A method according to aspect 41 or any other aspect herein wherein moving the mover in the first direction to the second position comprises rotating the mover about a first axis of the mover in the first direction to the second position.
   43. A method according to aspect 42 or any other aspect herein wherein an angular difference between the first position and the second position is between approximately 5° and 70°.
   44. A skin measuring apparatus according to aspect 42 or any other aspect herein wherein an angular difference between the first position and the second position is between approximately 10° and 25°.
   45. A method according to any one of aspects 42 to 44 or any other aspect herein wherein moving the mover in the first direction to the second position comprises actuating a rotary actuator connected to the mover.
   46. A method according to any one of aspects 42 to 45 or any other aspect herein wherein rotating the mover in the first direction to achieve the second position of the mover comprises rotating the mover in the first direction until a latching mechanism is engaged to prevent rotation of the mover in the second direction.
   47. A method according to any one of aspects 42 to 46 or any other aspect herein wherein the releasable catch is attached to a catch body, the catch body rotatable about the longitudinal axis of the mover and wherein measuring the force applied to the mover by the skin sample comprises measuring a force about the longitudinal axis of the mover applied to the catch body by the latch.
   48. A method according to aspect 41 or any other aspect herein wherein moving the mover in the first direction to the second position comprises translating the mover in the first direction to the second position.
   49. A method according to aspect 48 or any other aspect herein wherein moving the mover in the first direction to the second position comprises translating the mover in the first direction by actuation of an actuator or motor attached to the mover.
   50. A method according to any one of aspects 48 to 49 or any other aspect herein wherein a distance between the first position and the second position is between approximately 5 mm and 50 mm.
   51. A method according to any one of aspects 48 to 49 or any other aspect herein wherein a distance between the first position and the second position is between approximately 10 mm and 30 mm.
   52. A method according to any one of aspects 48 to 51 or any other aspect herein wherein the mover is translatably attached to the housing by one or more rails.
   53. A method according to any one of aspects 48 to 51 or any other aspect herein wherein the mover is translatably attached to the housing by one or more tracks.
   54. A method according to any one of aspects 41 to 53 or any other aspect herein, wherein setting the first position of the mover comprises releasably engaging a block with a latch fixed to the mover to hold the mover at the first position by abutting a first portion of the block with the housing and abutting of a second portion of the block with the latch.
   55. A method according to aspect 54 or any other aspect herein wherein abutting the second portion of the block with the latch comprises receiving the latch within a slot defined by the block.
   56. A method according to any one of 53 to 55 or any other aspect herein wherein abutting the first portion of the block with the housing comprising inserting at least a portion of the block into an aperture defined by the housing.
   57. A method according to any one of aspects 41 to 56 or any other aspect herein wherein adhering a first end of the mover to a skin sample comprises employing an adhesive to adhere the first end of the mover to the skin sample.
   58. A method according to any one of aspects 41 to 57 or any other aspect herein wherein moving the mover in the first direction to the second position comprises engaging a latching mechanism by engaging a latch fixed to the mover with a catch fixed to the housing.
   59. A method according to aspect 58 or any other aspect herein wherein engaging the latching mechanism comprises engaging a latch fixed to the mover with a catch fixed to the housing by resiliently deforming one or more of the latch and the catch.
   60. A method according to any one of aspects 58 and 59 or any other aspect herein wherein engaging the latching mechanism comprises engaging a latch fixed to the mover with a catch fixed to the housing by restoratively deforming one or more of the latch and the catch.
   61. A method according to any one of aspects 58 to 60 or any other aspect herein wherein releasing the mover to allow the mover to move in the second direction comprises resiliently deforming the catch to thereby release the latch by applying an external force to a trigger attached to the catch.
   62. A method according to aspect 61 or any other aspect herein wherein the trigger is accessible through an aperture defined by the housing.
   63. Methods comprising any features, combinations of features and/or sub-combinations of features described herein or inferable therefrom.
   64. Apparatus comprising any features, combinations of features and/or sub-combinations of features described herein or inferable therefrom.
   65. Kits comprising any features, combinations of features and/or sub-combinations of features described herein or inferable therefrom.

1. A skin measuring apparatus for measuring skin characteristics, the skin measuring apparatus comprising:
a mover attached to a housing to allow the mover to move in a first direction and a second direction relative to the housing;
a position sensor attached to the housing for measuring a position of the mover as the mover moves relative the housing between first and second positions;
wherein the position sensor is connected to measure the position when the mover freely moves relative to the housing;
a force sensor for measuring a force applied to the mover;
a latch fixed to the mover;
a releasable catch attached to the housing, the releasable catch engageable with the latch to prevent movement of the mover in the second direction from the second position until the releasable catch is released;
wherein the first direction is opposite the second direction;
wherein a first end of the mover is releasably attachable to a skin sample.

2. A skin measuring apparatus according to claim 1 wherein a mover attached to a housing to allow the mover to rotate about a first axis in the first direction and the second direction relative to the housing.

3. A skin measuring apparatus according to claim 2 wherein the mover is rotatable in the first direction and/or the second direction by actuation of a rotary actuator or motor attached to the mover.

4. A skin measuring apparatus according to claim 2 wherein the releasable catch is attached to a catch body, the catch body rotatable about the first axis.

5. A skin measuring apparatus according to claim 4 wherein the force sensor is fixed relative to the housing and a sensing portion of the force sensor is attached to the catch body to measure a torque about the first axis, the torque applied to the catch body by the latch.

6. A skin measuring apparatus according to claim 2 wherein an angular difference between the first position and the second position is between approximately 10° and 180°.

7. A skin measuring apparatus according to claim 2 wherein an angular difference between the first position and the second position is between approximately 5° and 70°.

8. A skin measuring apparatus according to claim 2 wherein an angular difference between the first position and the second position is between approximately 10° and 25°.

9. A skin measuring apparatus according to claim 2 wherein the mover is rotatably attached to the housing by one or more bearings.

10. A skin measuring apparatus according to claim 2 wherein the mover is rotatably attached to the housing by one or more bushings.

11. A skin measuring apparatus according to claim 1 wherein mover attached to a housing to allow the mover to translate along a first axis in the first direction and the second direction relative to the housing.

12. A skin measuring apparatus according to claim 11 wherein the mover is translatable in the first direction and/or the second direction by actuation of an actuator or motor attached to the mover.

13. A skin measuring apparatus according to claim 11 wherein a distance between the first position and the second position is between approximately 5 mm and 50 mm.

14. A skin measuring apparatus according to claim 11 wherein a distance between the first position and the second position is between approximately 10 mm and 30 mm.

15. A skin measuring apparatus according to claim 11 wherein the mover is translatably attached to the housing by one or more rails.

16. A skin measuring apparatus according to claim 11 wherein the mover is translatably attached to the housing by one or more tracks.

17. A skin measuring apparatus according to claim 1 wherein a sensing portion of the force sensor is fixed to the mover.

18. A skin measuring apparatus according to claim 1 wherein the latch engages the releasable catch by resilient deformation of one or more of the latch and the catch.

19. A skin measuring apparatus according to claim 1 wherein the latch engages the releasable catch by restorative deformation of one or more of the latch and the catch.

20. A method for measuring skin characteristics, the method comprising:
providing a mover attached to a housing to allow the mover to move in a first direction and a second direction relative to the housing;
setting a first position of the mover;
adhering a first end of the mover to a skin sample;
moving the mover in the first direction to a second position;
releasing the mover to allow the mover to move in the second direction;
measuring a force applied to the mover by the skin sample;
measuring the position of the mover while it moves in the first and second directions;
determining one or more skin characteristics based at least in part on the measured force and the measured angular position.
